SEQUENCE LISTING (34 sequences)

SEQ ID NO: 1 - protein, 116 aa, synthetic construct
DVQLVESGGGLVQAGGSLRLSCRASGHTFSDTAVGWFRRVPGKEREFVATIAGSGSIRYSESVKGRFTISGDNAKNMVYLQMNRLKPEDTGVYYCAAAVGISYDYWGQGTQVTVSS

SEQ ID NO: 2 - protein, 125 aa, synthetic construct
DVQLVESGGGLVQAGDSLRLSCVASGRTVSGGVMGWFRQAPGKGREFVASIKWESGRTYYVDSLKDGFTISRDSPKNTVYLQMTNLKPEDTAVYYCAAGIRVWQLSTLESTYDDWGQGTQVTVSS

SEQ ID NO: 3 - protein, 116 aa, synthetic construct
DVQLVESGGGLVQPGGSLRLSCRASGHTFSDLSVGWFRRAPGKAREFVATTAWNGSIRYAESVKGRFTISRDNAANMMYLQMNSLKPEDTAIYYCAAAVGISYDYWGQGTQVTVSS

SEQ ID NO: 4 - protein, 8 aa, synthetic construct
GHTFSDTA

SEQ ID NO: 5 - protein, 8 aa, synthetic construct
GHTFSDLS

SEQ ID NO: 6 - protein, 8 aa, synthetic construct
GRTVSGGV

SEQ ID NO: 7 - protein, 12 aa, synthetic construct
TIAGSGSIRYSE

SEQ ID NO: 8 - protein, 12 aa, synthetic construct
TTAWNGSIRYAE

SEQ ID NO: 9 - protein, 13 aa, synthetic construct
SIKWESGRTYYVD

SEQ ID NO: 10 - protein, 10 aa, synthetic construct
AAAVGISYDY

SEQ ID NO: 11 - protein, 18 aa, synthetic construct
AAGIRVWQLSTLESTYDD

SEQ ID NO: 12 - protein, 8 aa, synthetic construct
  VARIANT 7: note = T or L
  VARIANT 8: note = A or S
GHTFSDXX

SEQ ID NO: 13 - skipped

SEQ ID NO: 14 - protein, 12 aa, synthetic construct
  VARIANT 2: note = I or T
  VARIANT 4: note = G or W
  VARIANT 5: note = S or N
  VARIANT 11: note = S or A
TXAXXGSIRYXE

SEQ ID NO: 15 - protein, 25 aa, synthetic construct
DVQLVESGGGLVQAGGSLRLSCRAS

SEQ ID NO: 16 - protein, 25 aa, synthetic construct
DVQLVESGGGLVQPGGSLRLSCRAS

SEQ ID NO: 17 - protein, 25 aa, synthetic construct
DVQLVESGGGLVQAGDSLRLSCVAS

SEQ ID NO: 18 - protein, 25 aa, synthetic construct
  VARIANT 14: note = A or P
DVQLVESGGGLVQXGGSLRLSCRAS

SEQ ID NO: 19 - protein, 25 aa, synthetic construct
  VARIANT 14: note = A or P
  VARIANT 16: note = D or G
  VARIANT 23: note = V or R
DVQLVESGGGLVQXGXSLRLSCXAS

SEQ ID NO: 20 - protein, 16 aa, synthetic construct
VGWFRRVPGKEREFVA

SEQ ID NO: 21 - protein, 16 aa, synthetic construct
VGWFRRAPGKAREFVA

SEQ ID NO: 22 - protein, 16 aa, synthetic construct
MGWFRQAPGKGREFVA

SEQ ID NO: 23 - protein, 16 aa, synthetic construct
  VARIANT 7: note = V or A
  VARIANT 11: note = E or A
VGWFRRXPGKXREFVA

SEQ ID NO: 24 - protein, 16 aa, synthetic construct
  VARIANT 1: note = M or V
  VARIANT 6: note = Q or R
  VARIANT 7: note = A or V
  VARIANT 11: note = G, E or A
XGWFRXXPGKXREFVA

SEQ ID NO: 25 - protein, 34 aa, synthetic construct
SVKGRFTISGDNAKNMVYLQMNRLKPEDTGVYYC

SEQ ID NO: 26 - protein, 34 aa, synthetic construct
SVKGRFTISRDNAANMMYLQMNSLKPEDTAIYYC

SEQ ID NO: 27 - protein, 34 aa, synthetic construct
SLKDGFTISRDSPKNTVYLQMTNLKPEDTAVYYC

SEQ ID NO: 28 - protein, 34 aa, synthetic construct
  VARIANT 10: note = R or G
  VARIANT 14: note = K or A
  VARIANT 17: note = V or M
  VARIANT 23: note = R or S
  VARIANT 30: note = A or G
  VARIANT 31: note = V or I
SVKGRFTISXDNAXNMXYLQMNXLKPEDTXXYYC

SEQ ID NO: 29 - protein, 34 aa, synthetic construct
  VARIANT 2: note = L or V
  VARIANT 4: note = D or G
  VARIANT 5: note = G or R
  VARIANT 10: note = R or G
  VARIANT 12: note = S or N
  VARIANT 13: note = P or A
  VARIANT 14: note = K or A
  VARIANT 16: note = T or M
  VARIANT 17: note = V or M
  VARIANT 22: note = T or N
  VARIANT 23: note = N, R or S
  VARIANT 30: note = A or G
  VARIANT 31: note = V or I
SXKXXFTISXDXXXNXXYLQMXXLKPEDTXXYYC

SEQ ID NO: 30 - protein, 11 aa, synthetic construct
WGQGTQVTVSS

SEQ ID NO: 31 - protein, 161 aa, unidentified
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 32 - protein, 149 aa, synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSP

SEQ ID NO: 33 - protein, 5 aa, synthetic construct
LPXTG

SEQ ID NO: 34 - protein, 5 aa, synthetic construct
LPETG

(19) United States
(12) Patent Application Publication
(10) Pub. No.: US 2026/0102527 A1
(43) Pub. Date: Apr. 16, 2026

(54) CD8b-BINDING POLYPEPTIDES

(71) Applicant: VRIJE UNIVERSITEIT BRUSSEL, Brussel (BE)

(72) Inventors: Jo VAN GINDERACHTER, Brussel (BE); Geert RAES, Brussel (BE); Cécile VINCKE, Brussel (BE); Nick DEVOOGDT, Brussel (BE); Timo DE GROOF, Brussel (BE); Yoline LAUWERS, Brussel (BE)

(21) Appl. No.: 19/151,092

(22) PCT Filed: Jan. 26, 2024
(86) PCT No.: PCT/EP2024/051929

§ 371 Date: Jul. 25, 2025

(30) Foreign Application Priority Data
Jan. 27, 2023 (EP) 23153689.7
Aug. 28, 2023 (EP) 23193722.8

(51) Int. Cl. A61K 51/10; A61K 39/00; C07K 1/107; C07K 16/28

(52) U.S. Cl. CPC A61K 51/1027; C07K 1/1077; C07K 16/2815; A61K 2039/505; A61K 2123/00; C07K 2317/565; C07K 2317/567; C07K 2317/569; C07K 2317/92; C07K 2317/94

(57) ABSTRACT

The invention relates to polypeptides, in particular polypeptides comprising an immunoglobulin domain, binding to human and cynomolgus CD8b protein and to applications of such polypeptides such as for use as diagnostic agent, for example as an immunotracer.

### STATEMENT ON FUNDING

The project leading to this application has received funding from the Innovative Medicines Initiative 2 Joint Undertaking under Grant Agreement n^{o} 831514. This Joint Undertaking receives the support from the European Union's Horizon 2020 research and innovation programme and EFPIA.

### FIELD OF THE INVENTION

The invention relates to polypeptides, in particular polypeptides comprising an immunoglobulin domain, binding to human and cynomolgus CD8b protein and to applications of such polypeptides such as for use as diagnostic agent, for example as an immunotracer.

### BACKGROUND

Immunomodulating agents, and immune checkpoint inhibitors (ICIs) in particular, have revolutionized cancer treatment. Although highly effective in a subset of cancers and cancer patients, ICIs remain ineffective in a relative large fraction of cancers and cancer patients. Many efforts are now devoted to understanding the underlying inferior response or non-response to ICI therapy, and to finding ways to break poor response to ICI therapy. Prediction or follow-up of response of a cancer patient to ICI therapy, or to therapy including an immunomodulating agent in general, prior to or during the therapy would be ideal both for the patient (in terms of helping in finding the best possible therapy) and in socio-economic terms (helping in optimal allocation of available healthcare funding). Therapy prediction or follow up, using invasive methods such as biopsies or blood biomarkers, has, however, so far proven to be difficult as they do not provide a complete overview of the tumor micro-environment and/or lack spatial information. Therefore, a valuable alternative would be to have non-invasive means for detecting response or non-response to therapy, i.e. a means for detecting therapeutic outcome, as early as possible after start of the therapy.

Non-invasive immune-monitoring imaging applicable for in vivo monitoring of immune responses provides a means for helping in detecting therapeutic outcome and in understanding reasons for response or non-response to a therapy of interest. Many different immune-monitoring imaging techniques are being pursued, one of which being antibody-based tracers as part of the group of molecular tracers (reviewed in e.g. McCarthy et al. 2020, Front Immunol 11:1067).

Cluster of differentiation protein 8 (CD8) is a transmembrane co-receptor of the T-cell receptor (TCR) and a marker of CD8+ T-cells or cytotoxic T-cells (CTLs). CD8 can be made up of two subunits being the CD8α (or CD8a) and CD8β (or CD8b) chains. CTLs play a pivotal role in fighting off cancer, and infiltration of tumors with CTLs is generally accepted as a good predictor of favorable therapy outcome (e.g. Liu et al. 2021, Theranostics 11:5365-5386). An anti-CD8a minibody tracer has been developed by ImaginAb and has recently entered clinical phase I studies (Farwell et al. 2022, J Nucl Med 63:720-726). Clearly, there is a need to expand the armamentarium for non-invasive immune-monitoring.

### SUMMARY OF THE INVENTION

The invention relates to polypeptides binding to CD8b, wherein the amino acid sequence of such polypeptides is comprising a CDR1 region, a CDR2 region, and a CDR3 region, wherein the CDR1, CDR2 and CDR3 regions are selected from those CDR1, CDR2 and CDR3 regions as present in an immunoglobulin variable domain (IVD) defined by SEQ ID NOs:1, 2 or 3 and as determined by the Kabat, Chothia, Martin, or IMTG method. In one embodiment, the CDR1 region is chosen from SEQ ID NO:12 and 13, the CDR2 region is chosen from SEQ ID NOs: 14 and 9, and the CDR3 region is chosen from SEQ ID NOs: 10 and 11. In a further embodiment, such polypeptides are further comprising at least an FR1, FR2, FR3, or FR4 region as present in an IVD, such as present in an IVD as defined by SEQ ID NOs:1, 2 or 3; in a particular embodiment thereto, the FR1 region is defined by SEQ ID NO:19, the FR2 region is defined by SEQ ID NO:24, the FR3 region is defined by SEQ ID NO:29, and the FR4 region is defined by SEQ ID NO:30.

In any of the above-defined polypeptides binding to CD8b, the referred-to CDR and/or FR regions can be humanized and/or the IVD is humanized.

Any of the above-defined polypeptides binding to CD8b may further comprise a functional moiety. In one embodiment, such functional moiety is a His-tag or a peptide motif recognized by a peptide ligase. In another embodiment, such functional moiety is a detectable moiety; in a particular embodiment thereto, the detectable moiety may be linked to a specific site comprised in the polypeptide.

The invention further relates to isolated nucleic acids encoding a polypeptide binding to CD8b as defined above; to vectors comprising such nucleic acid; and to host cells expressing polypeptide binding to CD8b as defined above, or comprising a nucleic acid encoding such polypeptide, or comprising a vector such nucleic acid.

The invention further relates to pharmaceutical compositions comprising a polypeptide binding to CD8b as defined above.

The invention also relates to polypeptides binding to CD8b as defined above, or pharmaceutical compositions comprising such polypeptides binding to CD8b as defined above, for use in diagnosis, for use in surgery, for use in therapy monitoring, or for use as an imaging agent.

The invention also relates to methods for producing a polypeptide binding to CD8b as defined above comprising:
expressing the polypeptide in a host cell as defined above, or synthetic manufacture of the polypeptide binding to CD8b;
purifying the expressed or synthesized polypeptide; and
optionally, coupling a detectable moiety to the purified polypeptide.

### DESCRIPTION TO THE FIGURES

FIG. 1. Binding of anti-human CD8β single domain antibodies to SUP-T1 lymphoma cells. Binding of different concentrations of anti-human CD8β single domain antibodies (22719, 22728 and 22772) or an irrelevant single domain antibody (Irr Nb) to T cell-lymphoma SUP-T1 cells. Binding was detected as mean fluorescent intensity (MFI) via the C-terminal HA-tag and a fluorescently labeled anti-HA antibody using flow cytometry.

FIG. 2. Binding of anti-human CD8β single domain antibodies to primary T cells. Binding of anti-human CD8β single domain antibodies (22719, 22728 and 22772) or an irrelevant single domain antibody (Irr Nb) to primary T cells obtained from a healthy donor. CD8+ T cells (CD45+, CD11b−, CD19−, CD3+, CD4−) were gated. Single domain antibody binding was detected via its C-terminal HA-tag and a fluorescently labeled anti-HA antibody. Single domain antibody binding on primary T cells of 3 independent donors was detected as mean fluorescent intensity (MFI). Data presented as mean±S.D. Statistical analyses were performed using one way ANOVA with Dunnett's post hoc test. ns, p>0.05; *, p<0.05; ****, p<0.0001.

FIG. 3. SPECT/CT imaging of ^{99m}Tc-anti-human CD8β single domain antibodies. Representative SPECT/CT images of C57BL/6 wildtype (WT) or human CD8 transgenic mice intravenously injected with ^{99m}Tc-anti-human CD8β single domain antibodies or irrelevant single domain antibody. The arrows highlight uptake of ^{99m}Tc-anti-human CD8β single domain antibodies in the lymph nodes, thymus, intestines and spleen.

FIG. 4. Ex vivo biodistribution analysis of human CD8β-targeting single domain antibodies in naïve animals. Ex vivo γ-counting of the isolated organs from naïve human CD8 transgenic C57BL/6 mice (A) and wildtype C57BL/6 mice (B) 80 min after injection with ^{99m}Tc-labeled human CD8β-targeting single domain antibodies or irrelevant single domain antibody (Irr Nb). Biodistribution of the single domain antibodies in three mice is shown and expressed as mean±SD of percentage of injected activity per gram of organ or tissue (% IA/g).

FIG. 5. Ex vivo biodistribution analysis of anti-human CD8β single domain antibodies in MC38-tumor bearing human CD8 transgenic C57BL/6 mice. A) Ex vivo γ-counting of the tumors from MC38-tumor bearing human CD8 transgenic C57BL/6 mice 60 min after injection with ^{99m}Tc-labeled anti-human CD8β single domain antibodies or irrelevant single domain antibody (Irr Nb). B) Tumor-to-blood ratios of the different ^{99m}Tc-labeled single domain antibodies, calculated by dividing the percentage activity per gram (% IA/g) tumor by the percentage of injected activity per gram blood. Data presented as mean±S.D. Statistical analyses were performed using one way ANOVA with Dunnett's post hoc test. **, p<0.01; **** p<0.000

FIG. 6. Alignment of amino acid sequences of extracellular parts of human CD8a (SEQ ID NO:29, amino acids 22-82 of hCD8a sequence defined by Genbank Accession No. NP 001139345.1) and of human CD8b (SEQ ID NO:30, amino acids 22-170 of hCD8b sequence defined by Genbank Accession No. KAI2524241.1). Alignment was generated with the Clustal Omega tool using the standard parameters (tool available via www.ebiac.uk); both proteins share 21.4% identity.

FIG. 7. Binding of anti-human CD8 single domain antibodies to primary T cells and NK cells. (A-B) Binding of anti-human CD8a single domain antibody (R3HCD27), anti-human CD8β single domain antibodies (22719 and 22728) or an irrelevant single domain antibody (Irr Nb) to primary T cells and NK cells obtained from a healthy donor. T cells (CD45+, CD11b−, CD19−, CD3+) or NK cells (T cells (CD45+, CD11b−, CD19−, CD3−, CD56+) were gated. Single domain antibody binding was detected via its C-terminal HA-tag and a fluorescently labeled anti-HA antibody. Single domain antibody binding on different immune cell populations of 1 donor was detected as percentage of the immune cell population (A) or mean fluorescent intensity (MFI) (B).

FIG. 8. Human CD8β-targeting single domain antibodies do not induce cytotoxicity. (A) Histogram plots of human CD69-expression on CD8+ T-cells without treatment (“No Nb”) or overnight incubation of CD3/CD28 dynabeads or GLP-grade irrelevant single domain antibody (“Irr Nb”), anti-human CD8β single domain antibody 22728 (“22728”) or anti-human CD8β single domain antibody 22719 (“22719”) to human primary peripheral blood mononuclear cells. (B) Quantification of human CD69+CD8+ T-cells without treatment (“No Nb”) or overnight incubation of CD3/CD28 dynabeads or GLP-grade irrelevant single domain antibody (“Irr Nb”), anti-human CD8β single domain antibody 22728 (“22728”) or anti-human CD8β single domain antibody 22719 (“22719”). (C) Quantification of secreted IFN-γ levels in the supernatant of human primary peripheral blood mononuclear cells without treatment (“No Nb”) or overnight incubation of CD3/CD28 dynabeads or GLP-grade irrelevant single domain antibody (“Irr Nb”), anti-human CD8β single domain antibody 22728 (“22728”) or anti-human CD8β single domain antibody 22719 (“22719”) via ELISA. (D-E) Stimulation indices of IFN-γ (D) and IL-5 (E) of 30 healthy donors in the DC/CD4+ T-cell restimulation assays after incubation with non-immunogenic bevacizumab (“BVZ”), immunogenic keyhole limpet hemocyanin (“KLH”), GLP-grade irrelevant single domain antibody (“Irr Nb”), anti-human CD8β single domain antibody 22728 (“22728”) or anti-human CD8β single domain antibody 22719 (“22719”). Stimulation index indicates the amount of positive cell compared to untreated cells. All data presented as mean±S.D. Statistical analyses were performed using one way ANOVA with Dunnett's post hoc test. ns, p>0.05; ***, p<0.001; ****, p<0.0001.

FIG. 9. SPECT/CT imaging of ^{99m}Tc-Human CD8β-targeting single domain antibodies in SUP-T1 tumors. A) Representative SPECT/CT images and transverse sections of SUP-T1 tumor bearing mice intravenously injected with ^{99m}Tc-labeled irrelevant single domain antibody (“^{99m}Tc-Irr Nb”) or anti-human CD8β single domain antibody 22728 (“^{99m}Tc-22728”) or anti-human CD8β single domain antibody 22719 (“^{99m}Tc-22719”). B) Ex vivo γ-counting of the isolated tumors 80 min after injection with ^{99m}Tc-labeled single domain antibodies. Uptake of the single domain antibodies is expressed as injected activity per gram of tumor (% IA/g). Data presented as mean±S.D. Statistical analyses were performed using one way ANOVA with Dunnett's post hoc test. ***, p<0.001; ****, p<0.0001.

FIG. 10. Ex vivo biodistribution analysis of human CD8β-targeting single domain antibodies in SUP-T1 tumor bearing mice. Ex vivo γ-counting of the isolated organs from SUP-T1 tumor bearing nude mice 80 min after injection with ^{99m}Tc-labeled human CD8β-targeting single domain antibodies (“22728” and “22719”) or irrelevant single domain antibody (“Irr Nb”). Biodistribution of the single domain antibodies in seven mice is shown and expressed as mean±SD of percentage of injected activity per gram of organ or tissue (% IA/g). For each of the 21 organs on the X-axis are indicated from left to right the same 3 conditions: (1) [^{99m}Tc]Tc-Irr Nb (left-most), (2) [^{99m}Tc]Tc-23728 anti-hCD8β-targeting single domain antibody 23728, (3) [^{99m}Tc]Tc-22719 anti-hCD8β-targeting single domain antibody 22719 (right-most).

FIG. 11. ⁶⁸Ga-labeled human CD8β-targeting single domain antibodies are stable in injection buffer and human serum. Radiochemical purity of the ⁶⁸Ga-labeled anti-hCD8β-targeting single domain antibodies 22719 and 22728 in injection buffer (0.9% NaCl+5 mg/ml Vitamin C) at room temperature (RT) (A), injection buffer at 37° C. (B) or human serum (C). Purity is determined via radio-size exclusion chromatography and normalized to timepoint t=0.

FIG. 12. PET/CT imaging of ⁶⁸Ga-human CD8β-targeting single domain antibodies. A) Representative PET/CT image and transverse sections of human (h)CD8 transgenic mice intravenously injected with ⁶⁸Ga-labeled irrelevant single domain antibody (“⁶⁸Ga-Irr Nb”) or anti-hCD8β-targeting single domain antibody 22719 (“⁶⁸Ga22719”). The arrows highlight uptake of ⁶⁸Ga-labeled single domain antibody 22719 in the lymph node and spleen. B-C) Ex vivo γ-counting of the isolated lymph nodes (B) or spleen (C) 80 min after injection with ⁶⁸Ga-labeled single domain antibodies. Uptake of the single domain antibodies is expressed as injected activity per gram.

FIG. 13. Ex vivo biodistribution analysis of 68Ga-labeled human CD8β-targeting single domain antibodies in naïve human CD8 transgenic mice. Ex vivo γ-counting of the isolated organs from naïve human CD8 transgenic mice 80 min after injection with ⁶⁸Ga-labeled human CD8β-targeting single domain antibody 22719 (“22719”) or irrelevant single domain antibody (“Irr Nb”). Biodistribution of the single domain antibodies in three mice is shown and expressed as mean±S.D. of percentage of injected activity per gram of organ or tissue (% IA/g). For each of the 19 organs on the X-axis are indicated Irr Nb (left) and anti-hCD8β-targeting single domain antibody 22719 (right).

FIG. 14. PET/CT imaging of CD19-CAR T cells using the ⁶⁸Ga-labeled anti-human CD8β single domain antibody 22719 tracer. A) Growth curves of CD19-negative K562 and CD19-expressing Nalm6 tumors. NSG mice were injected with CD19-targeting CAR-T cells at day 25 post inoculation. Data presented as mean±S.E.M. B) Representative PET/CT coronal sections of NSG mice intravenously injected with ⁶⁸Ga-labeled single domain antibody 22719. One day before imaging, mice were injected with PBS (Untreated) or human CD19-targeting CAR-T cells (CAR T-cell treated). The arrow highlights uptake of ⁶⁸Ga-labeled single domain antibody 22719 in the liver C) Representative PET/CT coronal sections of CAR-T cell treated NSG mouse intravenously injected with ⁶⁸Ga-labeled Nb 22719. CAR-T cells dynamics were visualized at day 26, day 29 and day 32. The arrow highlights uptake of ⁶⁸Ga-labeled single domain antibody 22719 in the liver.

### DETAILED DESCRIPTION TO THE INVENTION

For purposes of diagnostic or molecular imaging in vivo, the imaging agent must be able to arrive at its target with high efficiency. This requires a combination of small-enough size in order to be able to achieve sufficient tissue penetration, selective binding to the target in order to achieve a high signal/noise ratio at the target site, and low overall body retention or accumulation (as a consequence of elimination from the body; typically in liver or kidneys) to avoid sites of high background signal which negatively influence signals at the target site.

While the majority, if not all, attention in targeting CD8-positive (CD8+) cells focuses on targeting the CD8α subunit, there is little or no attention for targeting the CD8β subunit. In terms of selectivity or specificity of targeting CD8+ (immune) cells, there is a distinction between targeting CD8α or CD8β. It was reported that the CD8αβ heterodimer is exclusively occurring on CD8+ T-cells (CTLs) whereas the CD8αα homodimer is occurring on several immune cell types including CTLs, γδ T cells, natural killer (NK) cells, and dendritic cells (DCs) (Geng & Raghavan 2019, Proc Natl Acad Sci USA 116:17951-17956; and references cited therein). Targeting CD8β thus provides the advantage of providing selectivity or specificity towards CTLs.

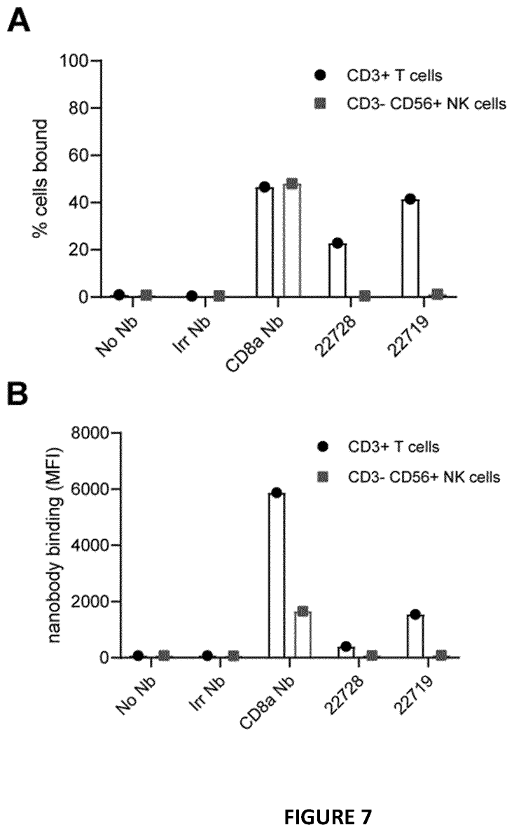

In work leading to the current invention, first of all a number of immunoglobulin single variable domain (ISVD) molecules, herein also referred to a single domain antibodies (sdAbs), binding with high specificity to human CD8b (hCD8b; CD8b used herein interchangeably with CD8β) and to cynomolgus CD8b were identified. The extracellular domain of hCD8b is only remotely related to the extracellular domain of hCD8a (CD8α) showing only 21.4% amino acid identity as calculated by the Clustal Omega tool (see FIG. 6). The CD8b ISVDs where shown to bind on cell-exposed CD8b but not to bind on cell-exposed CD8a. (FIG. 7).

The anti-CD8b sdAbs were identified after screening of llama immune libraries and were evaluated for binding (on CD8-expressing cells) and affinity using enzyme-linked immunosorbent assay (ELISA), flow cytometry and Surface Plasmon Resonance (SPR). Single photon emission computed tomography imaging in mice following intravenous injection of Technetium-99m (^{99m}Tc)-labelled or ⁶⁸Ga-labelled anti-CD8b sdAb revealed that these sdAbs have several properties making them interesting diagnostics: (i) good in vivo visualization of T-cell rich organs (lymph nodes, spleen, thymus and intestines) in healthy human CD8 transgenic mice (compared to an irrelevant ISVD), (ii) high uptake in tumors with a tumor-to-blood ratio significantly higher compared to an irrelevant ISVD, (iii) cross-reactivity with cynomolgous CD8b, and (iv) CTL selectivity. These combined characteristics render the identified CD8b-binding ISVDs well-suited as diagnostic agent, e.g. for molecular imaging.

Based hereon, the invention is defined in the following aspects and embodiments, and described in more detail hereafter. As the invention relates to polypeptides comprising complementarity determining regions (CDRs), some explanation is first provided on how such CDRs are determined.

The determination of the CDR regions in an antibody/immunoglobulin sequence generally depends on the algorithm/methodology applied (Kabat-, Chothia-, Martin (enhanced Chothia), IMGT (ImMunoGeneTics information system)-numbering schemes; see, e.g. http://www.bioinf.org.Uk/abs/index.html#kabatnum and http://www.imgt.org/IMGTScientificChart/Numbering/IMGTnumbering.html). Applying different methods to the same antibody/immunoglobulin sequence may give rise to different CDR amino acid sequences wherein the differences may reside in CDR sequence length and/or -delineation within the antibody/immunoglobulin/I(S)VD sequence. The CDRs of the CD8b-binding polypeptides of the invention can therefore be described as the CDR sequences as present in the single variable domain CD8b antibodies characterized herein, or alternatively as determined or delineated according to a well-known methodology such as according to the Kabat-, Chothia-, Martin (enhanced Chothia), or IMGT-numbering scheme or -method. The CDR sequences defined in SEQ ID NOs: 4-6, 7-9, and 10-11, for instance, have, been delineated from the CD8b single domain antibodies defined by SEQ ID NOs: 1-3 by means of the Kabat method. Applying another method may result in CDR sequences (slightly) different from those defined in SEQ ID NOs: 4-6, 7-9, and 10-11 (the FR sequences, see further, then differ accordingly).

In a first aspect, the invention relates to polypeptides specifically binding to CD8b (in particular to human and cynomolgus CD8b), wherein the amino acid sequence of the polypeptide is comprising a CDR1 region, a CDR2 region, and a CDR3 region, wherein the CDR1, CDR2 and CDR3 regions are selected from those CDR1, CDR2 and CDR3 regions, respectively, as present in any of CD8b-binding single domain antibodies or immunoglobulin variable domains (IVDs) or immunoglobulin single variable domains (ISVDs) defined by SEQ ID NOs:1, 2, or 3.

In particular, the polypeptides specifically binding to CD8b (in particular to human and cynomolgus CD8b) comprise an immunoglobulin (single) variable domain conveying specificity of the polypeptide for binding to CD8b wherein the I(S)VD is comprising a CDR1 region, a CDR2 region, and a CDR3 region, wherein the CDR1, CDR2 and CDR3 regions are selected from those CDR1, CDR2 and CDR3 regions, respectively, as present in any of CD8b-binding single domain antibodies defined by SEQ ID NOs: 1, 2, or 3.

In an embodiment thereto, the CDR regions are determined by applying the Kabat, Chothia, Martin, or IMTG method to SEQ ID NOs: 1, 2, or 3. In a more specific embodiment, the CDR regions are determined by the Kabat method and further defined as a CDR1 region chosen from SEQ ID NOs: 4, 5 or 6; a CDR2 region chosen from SEQ ID NOs: 7, 8 or 9; and a CDR3 region chosen from SEQ ID NOs:10 or 11.

Given the high degree of similarity between individual CDR1 amino acid sequences, between individual CDR2 amino acid sequences, and between individual CDR3 amino acid sequences, any CD8b-binding polypeptide comprising any possible combination of CDR1-CDR2-CDR3 amino acid sequences (determined with any of the above-described methods) is herewith envisaged.

In a further embodiment, polypeptides specifically binding to CD8b (in particular to human and cynomolgus CD8b), such as polypeptides specifically binding to CD8b comprising an immunoglobulin (single) variable domain, are characterized by the amino acid sequence of the polypeptide or of the I(S)VD comprising a CDR1 region defined by SEQ ID NO:12 or 13, a CDR2 region defined by SEQ ID NO:14 or 9, and a CDR3 region defined by SEQ ID NO:10 or 11.

In a further embodiment, polypeptides specifically binding to CD8b (in particular to human and cynomolgus CD8b), such as polypeptides specifically binding to CD8b (in particular to human and cynomolgus CD8b) comprising an immunoglobulin (single) variable domain, are characterized by comprising:
a) a CDR1 region defined by SEQ ID NO:13, a CDR2 region defined by SEQ ID NO:14, and a CDR3 region defined by SEQ ID NO:10 or 11; or
b) a CDR1 region defined by SEQ ID NO:12, a CDR2 region defined by SEQ ID NO:14, and a CDR3 region defined by SEQ ID NO:10 or 11; or
c) a CDR1 region defined by SEQ ID NO:12, a CDR2 region defined by SEQ ID NO:14, and a CDR3 region defined by SEQ ID NO:10; or
d) a CDR1 region defined by SEQ ID NO:13, a CDR2 region defined by SEQ ID NO:7, 8 or 9, and a CDR3 region defined by SEQ ID NO:10 or 11; or
e) a CDR1 region defined by SEQ ID NO:12, a CDR2 region defined by SEQ ID NO:7, 8 or 9, and a CDR3 region defined by SEQ ID NO:10 or 11; or
f) a CDR1 region defined by SEQ ID NO:4, 5 or 6, a CDR2 region defined by SEQ ID NO:14, and a CDR3 region defined by SEQ ID NO:10 or 11; or
g) a CDR1 region defined by SEQ ID NO:4 or 5, a CDR2 region defined by SEQ ID NO:7 or 8, and a CDR3 region defined by SEQ ID NO:10; or
h) a CDR1 region defined by SEQ ID NO:4, a CDR2 region defined by SEQ ID NO:7, and a CDR3 region defined by SEQ ID NO:10; or
i) a CDR1 region defined by SEQ ID NO:5, a CDR2 region defined by SEQ ID NO:8, and a CDR3 region defined by SEQ ID NO:10; or
j) a CDR1 region defined by SEQ ID NO:6, a CDR2 region defined by SEQ ID NO:9, and a CDR3 region defined by SEQ ID NO:11.

In a further embodiment, polypeptides specifically binding to CD8b (in particular to human and cynomolgus CD8b), such as polypeptides specifically binding to CD8b (in particular to human and cynomolgus CD8b) comprising an immunoglobulin (single) variable domain (I(S)VD), are characterized by further comprising at least a framework region (FR) such as a framework region from an immunoglobulin (single) variable domain (such as a FR region as present in an IVD as defined by SEQ ID NOs:1, 2 or 3), wherein the I(S)VD polypeptide can comprise up to 4 FR regions (FR1 preceding CDR1; FR2 interspersed between CDR1 and CDR2; FR3 interspersed between CDR2 and CDR3; FR4 following CDR3; wherein the relative positioning referred to is from the amino- to carboxy-terminus of the I(S)VD). FR1 regions can be selected from SEQ ID NOs:15 to 19. FR2 regions can be selected from SEQ ID NOs: 20 to 24. FR3 regions can be selected from SEQ ID NOs:25 to 29. The FR4 region can be defined by SEQ ID NO:30. Herein the sequence-defined FR regions are delineated based on the delineation of the respective CDR regions as determined according to the Kabat method; these FR regions thus can slightly differ in case the CDR regions are determined according to a non-Kabat method.

In a further embodiment, any of the above polypeptides specifically binding to CD8b (in particular to human and cynomolgus CD8b), such as any of the above polypeptides specifically binding to CD8b (in particular to human and cynomolgus CD8b) comprising an immunoglobulin (single) variable domain, are characterized by comprising:
k) a FR1 region defined by SEQ ID NO:19, a FR2 region defined by SEQ ID NO:24, a FR3 region defined by SEQ ID NO:29, and a FR4 region defined by SEQ ID NO:30; or
l) a FR1 region defined by SEQ ID NO:18, a FR2 region defined by SEQ ID NO:23, a FR3 region defined by SEQ ID NO:28, and a FR4 region defined by SEQ ID NO:30; or
m) a FR1 region defined by SEQ ID NO:19, a FR2 region defined by SEQ ID NO:20, 21 or 22, a FR3 region defined by SEQ ID NO:28 or 29, and a FR4 region defined by SEQ ID NO:30; or
n) a FR1 region defined by SEQ ID NO:19, a FR2 region defined by SEQ ID NO:23 or 24, a FR3 region defined by SEQ ID NO:25, 26, or 27, and a FR4 region defined by SEQ ID NO:30; or
o) a FR1 region defined by SEQ ID NO:19, a FR2 region defined by SEQ ID NO:20, 21 or 22, a FR3 region defined by SEQ ID NO:25, 26, or 27, and a FR4 region defined by SEQ ID NO:30; or
p) a FR1 region defined by SEQ ID NO:18, a FR2 region defined by SEQ ID NO:20, 21, or 22, a FR3 region defined by SEQ ID NO:28 or 29, and a FR4 region defined by SEQ ID NO:30; or
q) a FR1 region defined by SEQ ID NO:18, a FR2 region defined by SEQ ID NO:23 or 24, a FR3 region defined by SEQ ID NO:25, 26, or 27, and a FR4 region defined by SEQ ID NO:30; or
r) a FR1 region defined by SEQ ID NO:18, a FR2 region defined by SEQ ID NO:20, 21 or 22, a FR3 region defined by SEQ ID NO:25, 26, or 27, and a FR4 region defined by SEQ ID NO:30; or
s) a FR1 region defined by SEQ ID NO:15, a FR2 region defined by SEQ ID NO:20, a FR3 region defined by SEQ ID NO:25, and a FR4 region defined by SEQ ID NO:30; or
t) a FR1 region defined by SEQ ID NO:16, a FR2 region defined by SEQ ID NO:21, a FR3 region defined by SEQ ID NO:26, and a FR4 region defined by SEQ ID NO:30; or
u) a FR1 region defined by SEQ ID NO:17, a FR2 region defined by SEQ ID NO:22, a FR3 region defined by SEQ ID NO:27, and a FR4 region defined by SEQ ID NO:30.

In a further embodiment, any of the above polypeptides specifically binding to CD8b (in particular to human and cynomolgus CD8b), such as any of the above polypeptides specifically binding to CD8b (in particular to human and cynomolgus CD8b) comprising an immunoglobulin (single) variable domain, are characterized by comprising any of the above-defined combinations of CDR1, CDR2, and CD3 regions, and any of the above-defined combinations of FR1, FR2, FR3, and FR4 regions.

Some specific such combinations of CDR1, CDR2, and CD3 regions with combinations of FR1, FR2, FR3, and FR4 regions include (non-exhaustive):
v) CDR1 defined by SEQ ID NO:13, CDR2 defined by SEQ ID NO:14, CDR3 defined by SEQ ID NO:10 or 11, FR defined by SEQ ID NO:19, FR2 defined by SEQ ID NO:24, FR3 defined by SEQ ID NO:29, and FR4 defined by SEQ ID NO:30; or
w) CDR1 defined by SEQ ID NO:13, CDR2 defined by SEQ ID NO:9, CDR3 defined by SEQ ID NO:11 or 11, FR defined by SEQ ID NO:19, FR2 defined by SEQ ID NO:24, FR3 defined by SEQ ID NO:29, and FR4 defined by SEQ ID NO:30; or
x) CDR1 defined by SEQ ID NO:12, CDR2 defined by SEQ ID NO:14, CDR3 defined by SEQ ID NO:10, FR defined by SEQ ID NO:18, FR2 defined by SEQ ID NO:23, FR3 defined by SEQ ID NO:28, and FR4 defined by SEQ ID NO:30; or
y) CDR1 defined by SEQ ID NO:4, CDR2 defined by SEQ ID NO:7, CDR3 defined by SEQ ID NO:10, FR defined by SEQ ID NO:18, FR2 defined by SEQ ID NO:24, FR3 defined by SEQ ID NO:28, and FR4 defined by SEQ ID NO:30; or
z) CDR1 defined by SEQ ID NO:4, CDR2 defined by SEQ ID NO:7, CDR3 defined by SEQ ID NO:10, FR defined by SEQ ID NO:15, FR2 defined by SEQ ID NO:20, FR3 defined by SEQ ID NO:25, and FR4 defined by SEQ ID NO:30; or
aa) CDR1 defined by SEQ ID NO:5, CDR2 defined by SEQ ID NO:8, CDR3 defined by SEQ ID NO:10, FR defined by SEQ ID NO:18, FR2 defined by SEQ ID NO:24, FR3 defined by SEQ ID NO:28, and FR4 defined by SEQ ID NO:30; or
bb) CDR1 defined by SEQ ID NO:5, CDR2 defined by SEQ ID NO:8, CDR3 defined by SEQ ID NO:10, FR defined by SEQ ID NO:16, FR2 defined by SEQ ID NO:21, FR3 defined by SEQ ID NO:26, and FR4 defined by SEQ ID NO:30; or
cc) CDR1 defined by SEQ ID NO:6, CDR2 defined by SEQ ID NO:9, CDR3 defined by SEQ ID NO:11, FR defined by SEQ ID NO:17, FR2 defined by SEQ ID NO:22, FR3 defined by SEQ ID NO:27, and FR4 defined by SEQ ID NO:30.

In any of the above relating to the FR3 region, the lysine at position 14 in the FR3 region of SEQ ID NOs: 25 or 27 (positions 75 and 76 in SEQ ID NOs:1 and 2, respectively) can be changed into alanine without altering binding affinity but useful for conjugation to NOTA-chelator (see further) or other imaging moieties. In principle, any lysine residue in any of the FR regions of any of the herein described CD8b binding I(S)VDs can be changed into alanine for this purposes, on the condition that binding affinity is not significantly affected.

In a further embodiment, any of the above polypeptides specifically binding to CD8b (in particular to human and cynomolgus CD8b), such as any of the above polypeptides specifically binding to CD8b (in particular to human and cynomolgus CD8b) comprising an immunoglobulin (single) variable domain, are characterized by further comprising a moiety extending the half-life of the polypeptide once administered to a subject. Such half-life extending moiety can for instance be a serum albumin binding I(S)VD, or albumin itself. Other half-life extension modalities include PEGylation (or any modification such as glycol-PEGylation, biotinylated PEG), attaching (whether or not in the form of a fusion protein) peptides such as XTEN, PAS (“Pro Ala Ser”), ELP (elastin-like polypeptide), GLK (gelatin-like protein), HAPylation (adding (Gly4Ser)n peptide), and adding a polysaccharide moiety (reviewed in e.g. Zaman et al. 2019, J Controlled Release 301:176-189).

In any of the above, the CDR regions and/or FR regions and/or the I(S)VD may be humanized. Humanized CDRs and/or FRs and/or I(S)VDs can be obtained in any suitable manner known and thus are not strictly limited to polypeptides that have been obtained using a polypeptide that comprises a naturally occurring VHH domain as starting material. Humanized immunoglobulin single variable domains, may have several advantages, such as a reduced immunogenicity, compared to the corresponding naturally occurring VHH domains. Such humanization generally involves replacing one or more amino acid residues in the sequence of a naturally occurring CDR and/or framework region (FR) with the amino acid residues that occur at the same position in a human VH domain, such as a human VH3 domain. The humanizing substitutions should be chosen such that the resulting humanized immunoglobulin domains still retain the favourable properties of the originator immunoglobulin (or further improved by e.g. affinity maturation). The skilled person will be able to select humanizing substitutions or suitable combinations of humanizing substitutions, which optimize or achieve a suitable balance between the favourable properties provided by the humanizing substitutions on the one hand and the favourable properties of naturally occurring VHH domains on the other hand. In general, the specificity of binding to the target is not significantly (negatively) affected in a humanized antibody/immunoglobulin/(S)VD (or polypeptide comprising such antibody/immunoglobulin/(S)VD) and, in general, the affinity and/or avidity of binding to the target is not significantly (negatively) affected in a humanized antibody/immunoglobulin/(S)VD (or polypeptide comprising such antibody/immunoglobulin/(S)VD).

The CD8b-binding (in particular hCD8b- and cynomolgus CD8b-binding) polypeptides of the invention may comprise (in a fusion, conjugated therewith, or complexed therewith), one or more non-(poly)peptidic constituents such as detectable moieties (see further) or such as being pegylated (e.g. WO2017/059397), one or more further polypeptide(s) or polypeptide domain(s) such as e.g. a His-tag, or a peptide ligase motif such as a sortag motif (sortase peptide ligase amino acid substrate motif LPXTG (SEQ ID NO:33), e.g. LPETG (SEQ ID NO:34); Mao et al. 2004, J Am Chem Soc 126:2670-2671) or a peptide asparaginyl ligase motif (recognized by e.g. butelase 1 or VyPAL2; motif sequence being NXL wherein X can be e.g. Gly, Ser, Ala, Gln; Zhang et al. 2022, Int J Mol Sci 23:458; Hu et al. 2022, Plant Cell 34: 4936-4949). Such detectable moieties/tags/motifs are referred to herein as “functional moiety”. In one instance, the CD8b-binding polypeptide itself may be duplicated or multiplicated (wherein the monomers are e.g. connected through a flexible linker such as a linker based on Gly-Pro repeats, Pro-Ala repeats, Gly-Ser repeats, or combinations thereof) to form a multivalent (though monospecific) binding molecule. In another instance, the further polypeptide or polypeptide domain (connected through a flexible linker such as a linker based on Gly-Pro repeats, Pro-Ala repeats, Gly-Ser repeats, or combinations thereof, to the CD8b-binding polypeptide; or included in the CD8b binding polypeptide as a fusion protein) may confer increased serum half-life (e.g. a serum albumin binding protein or peptide; see above).

Thus, in any of the above, the CD8b-binding (in particular hCD8b- and cynomolgus CD8b-binding) polypeptide may further comprise a functional moiety. In one embodiment, the functional moiety is a detectable moiety. CD8b-binding (in particular hCD8b- and cynomolgus CD8b-binding) polypeptides as defined herein and carrying a detectable moiety therewith may be immunotracers; in case the detectable moiety is a radiolabel, the CD8b-binding (in particular hCD8b- and cynomolgus CD8b-binding) binding polypeptides may be radioimmunotracers.

Both bare CD8b-binding polypeptides (not comprising a detectable moiety) as described hereinabove and CD8b-binding polypeptides comprising a detectable moiety are useful when envisaging the in vivo imaging application. Indeed, bare CD8b-binding polypeptides may be co-administered with CD8b-binding polypeptides comprising a detectable moiety to a subject, or may be administered to a subject prior to administering CD8b-binding polypeptides comprising a detectable moiety, in order to mask the sink(s) of the CD8b-binding polypeptides, more in particular the kidney sink; as such sink background signals can be reduced. Moreover, it has been reported that preloading of unlabeled antibody may prolong the imaging window of the labeled antibodies (Nishio et al. 2020, Mol Imaging Biol 22:156-164).

A “detectable moiety” in general refers to a moiety that emits a signal or is capable of emitting a signal upon adequate stimulation, and is detectable by any means, preferably by a non-invasive means, once inside the human body. Furthermore, the detectable moiety may allow for computerized composition of an image, as such the detectable moiety may be called an imaging agent. Detectable moieties include fluorescence emitters, positron emitters, radioemitters, etc.

Measuring the amount of detectable moiety/imaging agent (comprised in, carried by, coupled to, chelated on a CD8b-binding polypeptide) is typically done with a device counting radioactivity or determining radiation (which can be of photonic nature) density or radiation concentration. The counted or determined radioactivity can be transformed into an image. Depending on the nature of the emission by the detectable moiety, it may be detectable by techniques such as PET (positron emission tomography), SPECT (single-photon emission computed tomography), fluorescence imaging, fluorescence tomography, near infrared imaging, near infrared tomography, optical tomography, etc.

Examples of radioemitters/radiolabels include ⁶⁸Ga, ^{110m}In, ¹⁸F, ⁴⁵Ti, ⁴⁴Sc, ⁴⁷Sc, ⁶¹Cu, ⁶⁰Cu, ⁶²Cu, ⁶⁶Ga, ⁶⁴Cu, ⁵⁵Ca, ⁷²As, ⁸⁶Y, ⁹⁰Y, ⁸⁹Zr, ¹²⁵I, ⁷⁴Br, ⁷⁵Br, ⁷⁶Br, ⁷⁷Br, ⁷⁸Br, ¹¹¹In, ^{114m}In, ¹¹⁴In, ^{99m}Tc, ¹¹C, ³²Cl, ³³Cl, ³⁴Cl, ¹²³I, ¹²⁴I, ¹³¹I, ¹⁸⁶Re, ¹⁸⁸Re, ¹⁷⁷Lu, ⁹⁹Tc, ²¹²Bi, ²¹³Bi, ²¹²Pb, ²²⁵Ac, ¹⁵³Sm, and ⁶⁷Ga. Fluorescence emitters include cyanine dyes (e.g. Cy5, Cy5.5, Cy7, Cy7.5), indolenine-based dyes, benzoindolenine-based dyes, phenoxazines, BODIPY dyes, rhodamines, Si-rhodamines, Alexa dyes, and derivatives of any thereof.

Many of the radionuclides have a metallic nature and are typically incapable of forming stable covalent bonds with proteins or peptides. One solution is to label proteins or peptides with radioactive metals by means of chelators, i.e. multidentate ligands, which form non-covalent compounds, called chelates, with the metal ions. A CD8b binding polypeptide may thus be coupled in any way to such chelator, which enables incorporation of a radionuclide; this allows a radionuclide to be coordinated, chelated or complexed to the CD8b-binding polypeptide. Chelators include polyaminopolycarboxylate-type chelators which can be macrocyclic or acyclic. A polyaminopolycarboxylate chelator can be conjugated to a CD8b-binding polypeptide e.g. via a thiol group of a cysteine residue or via an epsilon amine group of a lysine residue. Macrocyclic chelators for radioisotopes such as indium, gallium, yttrium, bismuth, radioactinides and radiolanthanides include DOTA (1,4,7,10-tetraazacyclododecane-1,4,7,10-tetraacetic acid) and derivatives thereof such as maleimidomonoamide-DOTA (1,4,7,10-tetraazacyclododecane-1,4,7-tris-acetic acid-10-maleimidoethylacetamide), DOTAGA (2,2′,2″-(10-(2,6-dioxotetrahydro-2H-pyran-3-yl)-1,4,7,10-tetraazacyclododecane-1,4,7-triyl)triacetic acid) with said polypeptide. Other chelators include NOTA (1,4,7-triazacyclononane-1,4,7-triacetic acid), and derivatives thereof such as NODAGA (2,2′-(7-(1-carboxy-4-((2,5-dioxopyrrolidin-1-yl)oxy)-4-oxobutyl)-1,4,7-triazonane-1,4-diyl)diacetic acid). Acyclic polyaminopolycarboxylate chelators include different derivatives of DTPA (diethylenetriamine-pentaacetic acid). Further chelating agents include DFO, CB-DO2A, 3p-C-DEPA, TCMC, Oxo-DO3A, TE2A, CB-TE2A, CB-TE1A1P, CB-TE2P, MM-TE2A, DM-TE2A, diamsar, NODASA, NETA, TACN-TM, 1B4M-DTPA, CHX-A″-DTPA, TRAP, NOPO, AAZTA, DATA, H2dedpa, H4octapa, H2azapa, H5decapa, H6phospa, HBED, SHBED, BPCA, CP256, PCTA, HEHA, PEPA, EDTA, TETA, and TRITA.

The detectable moiety in a CD8b-binding polypeptide, may itself be comprised in a prosthetic group and the prosthetic group may be linked to the polypeptide through a chelator or conjugating moiety such as a cyclooctyne comprising a reactive group that forms a covalent bond with an amine, carboxyl, carbonyl or thiol functional group on a CD8b-binding polypeptide. Cyclooctynes include dibenzocyclooctyne (DIBO), biarylazacyclooctynone (BARAC), dimethoxyazacyclooctyne (DIMAC) and dibenzocyclooctyne (DBCO), DBCO-PEG4-NHS-Ester, DBCO-Sulfo-NHS-Ester, DBCO-PEG4-Acid, DBCO-PEG4-Amine or DBCO-PEG4-Maleimide. An example of an ¹⁸F-labelled prosthetic group is ¹⁸F-3-(2-(2-(2-(2-azidoethoxy)ethoxy)ethoxy)ethoxy)-2-fluoropyridine (¹⁸F-FFPEGA). Other ¹⁸F-labelled prosthetic groups include N-Succinimidyl-4-[¹⁸F]fluorobenzoate ([⁸F]SFB) (e.g. Li et al. 2014, Applied Radiation and Isotopes 94:113-117); I-labelled prosthetic groups include N-succinimidyl 4-guanidinomethyl-3-[(*)I]iodobenzoate ([(*)I]SGMIB) and N-succinimidyl 3-guanidinomethyl-5-[(*)I]iodobenzoate (iso-[(*)I]SGMIB) wherein (*)I is for instance 131I (see e.g. Choi et al. 2014, Nucl Med Biol 41:802-812).

Conjugation methods as described above may result in heterogeneous tracer populations. Site-specific conjugation strategies try to overcome this shortcoming and include chemoenzymatic methods to couple polypeptides such as antibodies/immunoglobulins/I(S)VDs with a chelator or detectable moiety such as via sortase-mediated transpeptidation (Antos et al. 2009, Curr Protoc Protein Sci, Chapter 15:unti-15.3) (reviewed by e.g. Massa et al. 2016, Exp Opin Drug Deliv 13:1149-1163) or via peptide ligase mediated conjugation (see above). The CD8b-binding (in particular hCD8b- and cynomolgus CD8b-binding) polypeptides as described hereinabove thus may have the detectable moiety linked to a specific site comprised in the polypeptide, such as to form a homogeneous or quasi homogeneous population of tracer molecules.

Other aspects relate to isolated nucleic acids encoding a CD8b-binding (in particular hCD8b- and cynomolgus CD8b-binding) polypeptide as described hereinabove; to vectors comprising such nucleic acid; and to host cells comprising such nucleic acid or vector, and/or expressing a CD8b-binding (in particular hCD8b- and cynomolgus CD8b-binding) polypeptide as described hereinabove.

A further aspect relates to pharmaceutical compositions comprising a CD8b-binding (in particular hCD8b- and cynomolgus CD8b-binding) polypeptide as described hereinabove (CD8b-binding polypeptides without/not comprising a functional moiety, CD8b-binding polypeptides with/comprising a functional moiety, or CD8b-binding polypeptides with/comprising a detectable moiety). Such pharmaceutical compositions comprise a CD8b-binding (in particular hCD8b- and cynomolgus CD8b-binding) polypeptide as described hereinabove formulated in a excipient. The excipient is a suitable excipient, such as a pharmaceutically acceptable excipient, and is compatible with administration to a subject, e.g. is not toxic. On the other hand, the excipient may function in e.g. stabilizing or solubilizing the CD8b-binding polypeptide such as a CD8b-binding polypeptide with/comprising a functional moiety.

Yet a further aspect relates to a CD8b-binding (in particular hCD8b- and cynomolgus CD8b-binding) polypeptide as described hereinabove, or to a pharmaceutical composition comprising it for use in diagnosis, for use in surgery or in guiding surgery, for use in therapy monitoring, and in particular for use as an imaging agent such as described herein. Alternatively, the invention relates to methods of diagnosis or therapy monitoring, said methods comprising administration of a CD8b-binding (in particular hCD8b- and cynomolgus CD8b-binding) polypeptide as described hereinabove, or of a pharmaceutical composition comprising it, to a subject. As a result of the administration, the presence of CD8+ cells can be diagnosed or the fluctuation of such cells before, after start or during a therapy such as an immunomodulating therapy can be followed up. Alternatively, the invention relates to methods of surgical resection of a tumor, said methods comprising administration of a CD8b-binding (in particular hCD8b- and cynomolgus CD8b-binding) polypeptide as described hereinabove, or of a pharmaceutical composition comprising it, to a subject, wherein the CD8b binding polypeptide, especially when comprising a detectable moiety, can assist in delineating the tumor during resection. In a particular embodiment, the CD8b binding polypeptides as described hereinabove are applied in the field of cancer or tumor imaging, in the field of monitoring of cancer or tumor therapy, in the field of cancer or tumor diagnosis, or in the field of cancer or tumor surgery or guiding cancer or tumor surgery.

Specificity or selectivity of cell targeting, in particular CTL targeting via CD8b, refers to the situation in which a composition, at a certain concentration, is interacting (such as binding) with the intended target cell with higher efficacy (e.g. with an at least 2-fold, 5-fold, or 10-fold higher efficacy, or e.g. with at least 20-, 50- or 100-fold higher efficacy) than the efficacy with which the composition is interacting with other cells (not intended as target cell). Exclusivity of cell targeting refers to the situation in which a composition is interacting only with the intended target cell.

### Diagnosis

In general “diagnosis” herein refers to detection of CD8b or of cells displaying CD8b, such as human or cynomolgus CD8b. This can be ex vivo or in vitro such as in a sample from a (human) subject (and such as by for instance ELISA, immunocytochemistry (ICH), western blot, or surface Plasmon resonance). This can also be in vivo diagnosis, in particular non-invasive in vivo diagnosis such as by medical imaging or molecular imaging as described hereinabove. Diagnosis, whether on a sample from a (human) subject or by in vivo (imaging) methods allows to monitor response to therapy, such as response to immunotherapy or an immunomodulating therapy, such as therapy of a subject having a tumor or having cancer. Diagnosis, and especially imaging, may also assist in defining e.g. a tumour in need of surgical resection, thus in assisting surgery or guiding surgery.

### Therapy Monitoring

As examples of immunomodulating therapeutic compounds, the FDA has approval anti-PD-1 mAbs pembrolizumab, nivolumab and cemiplimab; anti-PD-L1 mAbs durvalumab, atezolizumab and avelumab; anti-CTLA4 mAb ipilimumab; and the combination of anti-LAG3 mAb relatlimab and nivolumab, which have since become available as standard-of-care for several cancer types. The downside of this success story is the high cost of such treatments, easily surpassing $100,000 per patient (e.g. Aguiar et al. 2017, Ann Oncol 28:2256-2263), and the observation that these immune checkpoint blockers are only of benefit for a subset of patients (e.g. Alsaab et al. 2017, Front Pharmacol 8:561). The failure rate, combined with the high cost for society, drives the search for predictive biomarkers that can help select the right treatment for the right patient. Currently the most commonly used predictive biomarker is PD-L1 expression assessed via IHC on tumor biopsies, although limitations are obviously present. Limitations such as heterogeneous expression, the role of expression outside of the tumor, and its dynamic expression during the disease process. Such limitations could be overcome by noninvasive molecular imaging using radiolabeled tracers that allow deep tumor penetration and repeated quantification of a reliable marker—this would enable mapping of primary tumors and metastatic lesions or of the immune landscape within such tumors or lesions both before and during the treatment.

Immunotracer-based tumor imaging in vivo can assist in disease diagnostics, patient stratification (determining which patients are more likely to respond to immunotherapy), disease monitoring (changes in the tumor images obtained during therapy reflect response or non-response to immunotherapy) and the design and development of new immunotherapies (throughout pre-clinical or clinical development). In particular, imaging (such as immunoPET imaging) of immune cells, in particular CD8+ immune cells, based on labeled anti-CD8b moieties of the current invention can likewise assist in monitoring the efficacy of immunotherapy, immunogenic or immunomodulating therapy, while also assisting in patient stratification and providing valuable information when designing and/or developing new immunotherapies, immunogenic therapies or immunomodulating therapies.

### Immunotherapy and Immunogenic Therapy

Immunotherapy in general is defined as a treatment that uses the body's own immune system to help fight a disease, more specifically cancer in the context of the current invention. Immunotherapeutic treatment as used herein refers to the reactivation and/or stimulation and/or reconstitution of the immune response of a mammal towards a condition such as a tumour, cancer or neoplasm evading and/or escaping and/or suppressing normal immune surveillance. The reactivation and/or stimulation and/or reconstitution of the immune response of a mammal in turn in part results in an increase in elimination of tumorous, cancerous or neoplastic cells by the mammal's immune system (anticancer, antitumour or anti-neoplasm immune response; adaptive immune response to the tumour, cancer or neoplasm). Immunotherapeutic agents of particular interest include immune checkpoint inhibitors (such as anti-PD-1, anti-PD-L1 or anti-CTLA-4 antibodies), bispecific antibodies bridging a cancer cell and an immune cell, dendritic cell vaccines, oncolytic viruses, cell-based therapies (e.g. CAR-T). Immunotherapy is a promising new area of cancer therapeutics and several immunotherapies are being evaluated pre-clinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumour progression. An overview of clinical developments in the field of immune checkpoint therapy is given by Fan et al. 2019 (Oncology Reports 41:3-14). Monoclonal antibodies targeting and inhibiting PD-1 include pembrolizumab, nivolumab, and cemiplimab. Monoclonal antibodies targeting and inhibiting PD-L1 include atezolizumab, avelumab, and durvalumab. Monoclonal antibodies targeting and inhibiting CTLA-4 include ipilimumab. Combinatorial cancer treatments that include chemotherapies can achieve higher rates of disease control by impinging on distinct elements of tumour biology to obtain synergistic antitumour effects. It is now accepted that certain chemotherapies can increase tumour immunity by inducing immunogenic cell death and by promoting escape in cancer immunoediting, such therapies are therefore called immunogenic therapies as they provoke an immunogenic response. Drug moieties known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187). Other forms of immunotherapy include chimeric antigen receptor (CAR) T-cell therapy in which allogeneic T-cells are adapted to recognize a tumour neo-antigen and oncolytic viruses preferentially infecting and killing cancer cells. Treatment with RNA, e.g. encoding MLKL, is a further means of provoking an immunogenic response (Van Hoecke et al. 2018, Nat Commun 9:3417), as well as vaccination with neo-epitopes (Brennick et al. 2017, Immunotherapy 9:361-371).

In a final aspect, the invention relates to methods for producing a CD8b-binding (in particular hCD8b- and cynomolgus CD8b-binding) polypeptide according to the invention, such methods comprising the steps of:
expressing the CD8b-binding (in particular hCD8b- and cynomolgus CD8b-binding) polypeptide in a suitable host cell (such as comprising a nucleic acid or vector as described herein, or synthetic manufacture of the CD8b-binding (in particular hCD8b- and cynomolgus CD8b-binding) polypeptide; and
purifying the expressed or synthesized/manufactured CD8b-binding (in particular hCD8b- and cynomolgus CD8b-binding) polypeptide.

Such methods may further comprise a step of coupling, incorporating, binding, ligating, bonding, complexing, chelating, conjugating (e.g. site-specifically conjugating) or otherwise linking, covalently or non-covalently, a detectable moiety to the purified CD8b-binding polypeptide.

### Other Definitions

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, a CDR defined in/by SEQ ID NO:X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X.

The term “antibody” as used herein, refers to an immunoglobulin (Ig) molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The term “immunoglobulin domain” as used herein refers to a globular region of an antibody chain (such as e.g., a chain of a conventional 4-chain antibody or a chain of a heavy chain antibody), or to a polypeptide that essentially consists of such a globular region. Immunoglobulin domains are characterized in that they retain the immunoglobulin fold characteristic of antibody molecules, which consists of a two-layer sandwich of about seven antiparallel β-strands arranged in two β-sheets, optionally stabilized by a conserved disulphide bond.

The specificity of an antibody/immunoglobulin/(S)VD for an antigen is defined by the composition of the antigen-binding domains in the antibody/immunoglobulin/(S)VD (usually one or more of the CDRs, the particular amino acids of the antibody/immunoglobulin/(S)VD interacting with the antigen forming the paratope) and the composition of the antigen (the parts of the antigen interacting with the antibody/immunoglobulin/(S)VD forming the epitope). Specificity of binding is understood to refer to a binding between an antibody/immunoglobulin/I(S)VD with a single target molecule or with a limited number of target molecules that (happen to) share an epitope recognized by the antibody/immunoglobulin/(S)VD.

Affinity of an antibody/immunoglobulin/I(S)VD for its target is a measure for the strength of interaction between an epitope on the target (antigen) and an epitope/antigen binding site in the antibody/immunoglobulin/(S)VD. It can be defined as:

$${K}_{A} = \frac{[ A b - A g ]}{[ A b ] [ A g ]}$$

Wherein KA is the affinity constant, [Ab] is the molar concentration of unoccupied binding sites on the antibody/immunoglobulin/(S)VD, [Ag] is the molar concentration of unoccupied binding sites on the antigen, and [Ab-Ag] is the molar concentration of the antibody-antigen complex.

Avidity provides information on the overall strength of an antibody/immunoglobulin/(S)VD-antigen complex, and generally depends on the above-described affinity, the valency of antibody/immunoglobulin/(S)VD and of antigen, and the structural interaction of the binding partners.

The term “immunoglobulin variable domain” (abbreviated as “I(S)VD”) as used herein means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Methods for delineating/confining a CDR in an antibody/immunoglobulin/(S)VD have been described hereinabove.

The term “immunoglobulin single variable domain” (abbreviated as “ISVD”), equivalent to the term “single variable domain”, defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associated) immunoglobulin domains such as light and heavy chain variable domains, i.e., by a VH-VL pair of immunoglobulin domains, which jointly bind to an epitope of the respective antigen. In contrast, immunoglobulin single variable domains are capable of specifically binding to an epitope of the antigen without pairing with an additional immunoglobulin variable domain. The binding site of an immunoglobulin single variable domain is formed by a single VH/VHH or VL domain. Hence, the antigen binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). In one embodiment of the invention, the immunoglobulin single variable domains are heavy chain variable domain sequences (e.g., a VH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a “dAb” or dAb (or an amino acid sequence that is suitable for use as a dAb) or a Nanobody® (as defined herein, and including but not limited to a VHH); other single variable domains, or any suitable fragment of any one thereof. In particular, the immunoglobulin single variable domain may be a Nanobody® (as defined herein) or a suitable fragment thereof. Note: Nanobody®, Nanobodies® and Nanoclone® are registered trademarks of Ablynx N.V. For a general description of Nanobodies®, reference is made to the further description below, as well as to the prior art cited herein, such as e.g. described in WO2008/020079.

“VHH domains”, also known as VHHs, VHH domains, VHH antibody fragments, and VHH antibodies, have originally been described as the antigen binding immunoglobulin (variable) domain of “heavy chain antibodies” (i.e., of “antibodies devoid of light chains”; Hamers-Casterman et al. 1993 (Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody®, reference is made to the review article by Muyldermans 2001 (Reviews in Molecular Biotechnology 74:277-302), as well as to the following patent applications, which are mentioned as general background art: WO 94/04678, WO 95/04079 and WO 96/34103; WO 94/25591, WO 99/37681, WO 00/40968, WO 00/43507, WO 00/65057, WO 01/40310, WO 01/44301, EP 1134231 and WO 02/48193; WO 97/49805, WO 01/21817, WO 03/035694, WO 03/054016 and WO 03/055527; WO 03/050531; WO 01/90190; WO 03/025020; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references, Nanobody® (in particular VHH sequences and partially humanized Nanobody®) can in particular be characterized by the presence of one or more “Hallmark residues” in one or more of the framework sequences. A further description of the Nanobody®, including humanization and/or camelization of Nanobody®, as well as other modifications, parts or fragments, derivatives or “Nanobody® fusions”, multivalent constructs (including some non-limiting examples of linker sequences) and different modifications to increase the half-life of the Nanobody® and their preparations can be found e.g. in WO 08/101985 and WO 08/142164.

“Domain antibodies”, also known as “dAbs” (the terms “Domain Antibodies” and “dAbs” being used as trademarks by the GlaxoSmithKline group of companies) have been described in e.g., EP 0368684, Ward et al. 1989 (Nature 341:544-546), Holt et al. 2003 (Tends in Biotechnology 21:484-490) and WO 03/002609 as well as for example WO 04/068820, WO 06/030220, and WO 06/003388. Domain antibodies essentially correspond to the VH or VL domains of non-camelid mammalians, in particular human 4-chain antibodies. In order to bind an epitope as a single antigen binding domain, i.e., without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutic use in humans. It should also be noted that single variable domains can be derived from certain species of shark (for example, the so-called “IgNAR domains”, see for example WO 05/18629).

Immunoglobulin single variable domains such as Domain antibodies and Nanobody® (including VHH domains and humanized VHH domains), can be subjected to affinity maturation by introducing one or more alterations in the amino acid sequence of one or more CDRs, which alterations result in an improved affinity of the resulting immunoglobulin single variable domain for its respective antigen, as compared to the respective parent molecule. Affinity-matured immunoglobulin single variable domain molecules of the invention may be prepared by methods known in the art, for example, as described by Marks et al. 1992 (Biotechnology 10:779-783), Barbas et al. 1994 (Proc Natl Acad Sci USA 91:3809-3813), Shier et al. 1995 (Gene 169:147-155), Yelton et al. 1995 (Immunol 155:1994-2004), Jackson et al. 1995 (J Immunol 154:3310-3319), Hawkins et al. 1992 (J Mol Biol 226:889-896), Johnson and Hawkins (Affinity maturation of antibodies using phage display, Oxford University Press, 1996). The process of designing/selecting and/or preparing a polypeptide, starting from an immunoglobulin single variable domain such as a Domain antibody or a Nanobody®, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted and examples of such formats for instance to avoid glycosylation will be clear to the skilled person based on the disclosure herein.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains that are as generally defined for in the previous paragraphs, but in which at least one amino acid residue is present (and in particular, at least one framework residue) that is and/or that corresponds to a humanizing substitution (as defined herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known perse, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized.

A “serum albumin binding agent”, or “serum albumin binding polypeptide”, as used herein, is a protein-based agent capable of specific binding to serum albumin. In various embodiments, the serum albumin binding agent may bind to the full-length and/or mature forms and/or isoforms and/or splice variants and/or fragments and/or any other naturally occurring or synthetic analogues, variants or mutants of serum albumin. In various embodiments, the serum albumin binding agent of the invention may bind to any forms of serum albumin, including monomeric, dimeric, trimeric, tetrameric, heterodimeric, multimeric and associated forms. In an embodiment, the serum albumin binding agent binds to the monomeric form of serum albumin. In an embodiment, the present serum albumin binding polypeptide comprises immunoglobulin variable domain with an antigen binding site that comprises three complementarity determining regions (CDR1, CDR2 and CDR3). In an embodiment said antigen binding site recognizes one or more epitopes present on serum albumin. In various embodiments, the serum albumin binding agent comprises a full length antibody or fragments thereof. In an embodiment, the serum albumin binding agent comprises a single domain antibody or an immunoglobulin single variable domain (ISVD). In a specific embodiment, the serum albumin binding agent binds to serum albumin of rat (Uniprot P02770). In a specific embodiment, the serum albumin binding agent binds to serum albumin of mouse (Uniprot P07724). In a specific embodiment, the serum albumin binding agent binds to human serum albumin (Uniprot P02768).

The aspects and embodiments described above in general may comprise the administration of a CD8b-binding (in particular hCD8b- and cynomolgus CD8b-binding) polypeptide or pharmaceutical composition comprising it to a mammal in need thereof, i.e., harbouring a tumour, cancer or neoplasm in need of (non-invasive) medical imaging, diagnosis, surgery (or guiding surgery) or therapy monitoring. In general an effective amount of the CD8b-binding (in particular hCD8b- and cynomolgus CD8b-binding) polypeptide or pharmaceutical composition comprising it is administered to the mammal in need thereof in order to meet the desired effect. The effective amount will depend on many factors such as route of administration and will need to be determined on a case-by-case basis by the physician. “Administering” means any mode of contacting that results in interaction between an agent (a CD8b-binding polypeptide as described herein) or composition comprising the agent (such as a medicament or pharmaceutical composition) and an object (e.g. cell, tissue, organ, body lumen) with which said agent or composition is contacted. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

The term “effective amount” refers to the dosing regimen of the agent (a CD8b-binding polypeptide as described herein) or composition comprising the agent (e.g. pharmaceutical composition). The effective amount will generally depend on and/or will need adjustment to the mode of contacting or administration. To obtain or maintain the effective amount, the agent or composition comprising the agent may be administered as a single dose or in multiple doses. The effective amount may further vary depending on the severity of the condition that needs to be diagnosed, imaged, or operated; this may depend on the overall health and physical condition of the mammal or patient and usually a doctor's or physician's assessment will be required to establish what is the effective amount. The effective amount may further be obtained by a combination of different types of contacting or administration.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims. The content of the documents cited herein are incorporated by reference.

### EXAMPLES

### Example 1. Anti-Human CD8b Single Domain Antibodies Bind Human CD8 with High Affinities and are Thermostable

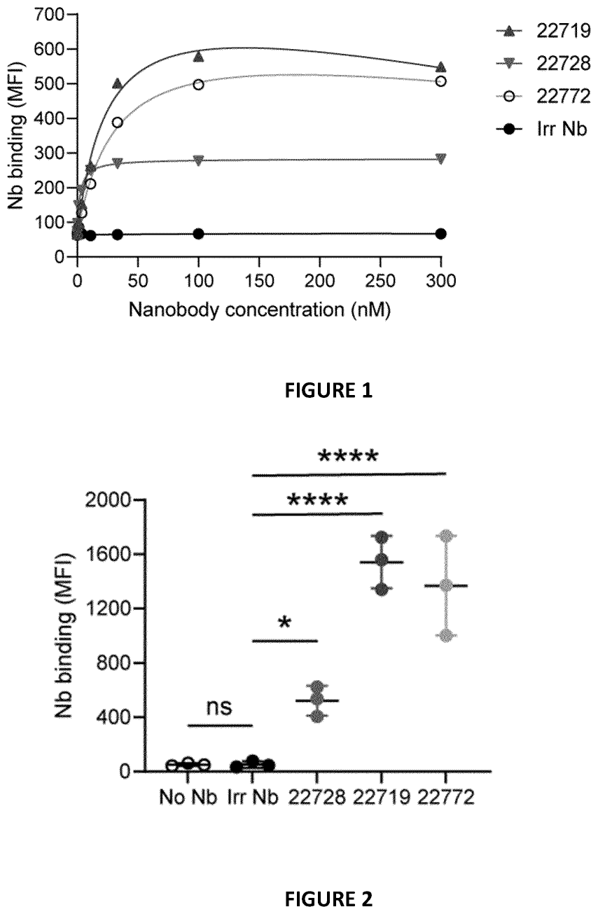

To identify single domain antibodies against the extracellular part of the human (h)CD8β chain, two llamas were immunized with recombinant hCD8β protein. Subsequent phage display panning and screening resulted in 175 unique single domain antibodies (Nbs) belonging to 33 different B cell lineages. Further screening of these single domain antibodies resulted in the selection of 3 anti-hCD8β single domain antibodies, namely Nb 22728, Nb 22719 and Nb 22772 with Nb 22719 and Nb 22772 being from the same B cell lineage (Table 1). All three anti-hCD8β single domain antibodies displayed low nanomolar binding affinity to the human CD8 protein as shown via surface plasmon resonance (Table 1). In line, similar binding affinities to SUP-T1 cells, a T cell lymphoma cell line that endogenously expresses human CD8, were seen in flow cytometry, while no binding was observed for an irrelevant single domain antibody, targeting the 5T2 multiple myeloma M protein (Lemaire et al. 2014, Leukemia 28:444-447) (FIG. 1 and Table 1). Interestingly, Nb 22719 and 22772 displayed higher binding plateaus compared to Nb 22728. Next, we determined the ability of the anti-hCD8β single domain antibodies to bind primary CD8+ T cells ex vivo (FIG. 2). While no binding was seen for the irrelevant single domain antibody, all three anti-hCD8β single domain antibodies were able to bind the CD8+ T cell population within a pool of primary T cells. In line with the previous results, higher binding plateaus were seen for Nb 22719 and 22772 compared to Nb 22728.

Moreover, the anti-hCD8β single domain antibodies did not show any binding to CD3− CD56+ NK cells, known to express hCD8α but not hCD8β (Geng & Raghavan 2019, Proc Natl Acad Sci USA 116:17951-17956) (FIG. 7A-B). In contrast, binding of an anti hCD8α-single domain antibody (clone R3HCD27 of US20190071500A1) to CD3− CD56+ NK cells was observed, indicating the enhanced specific binding of anti-hCD8β single domain antibodies to CD8+ T cells compared to hCD8α-targeting compounds.

As the extracellular part of the human and cynomolgus CD8β chain share high homology, we also assessed the binding affinity of the anti-hCD8β single domain antibodies to cynomolgus CD8β protein (Table 1). Equilibrium binding ELISA with the anti-hCD8β single domain antibodies indicated similar low nanomolar binding affinities to both human and cynomolgus CD8β protein for all three single domain antibodies.

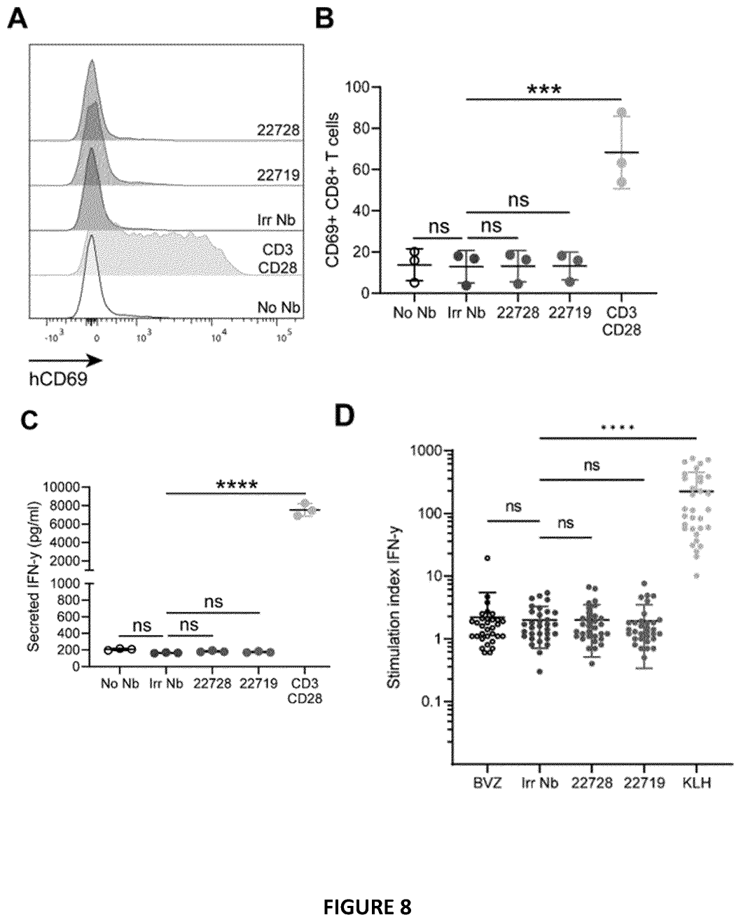

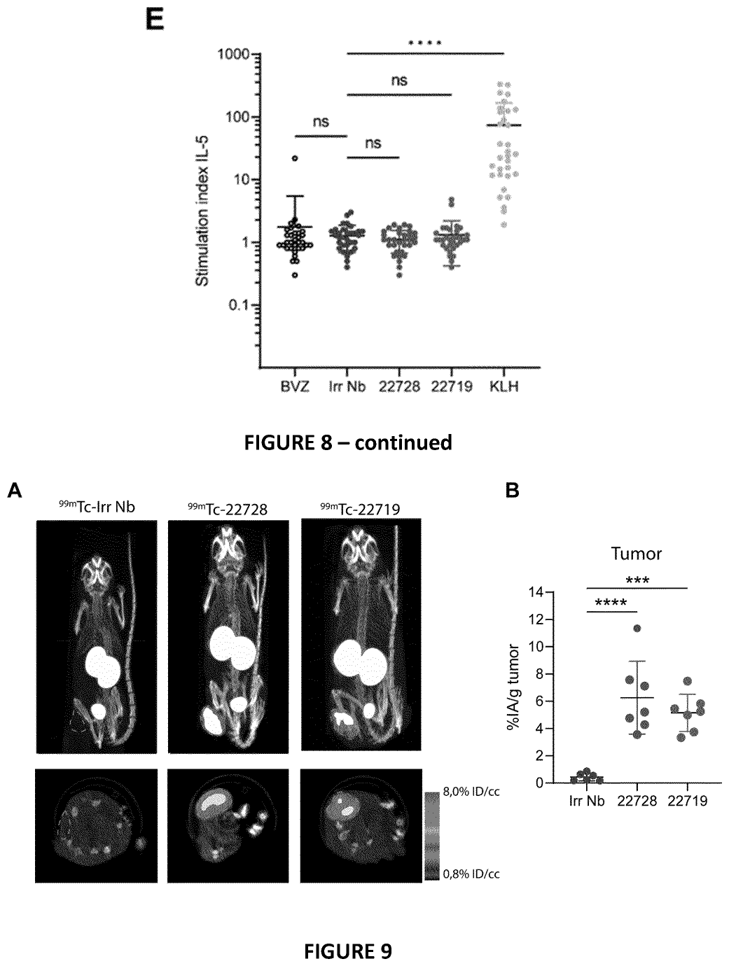

As binding of the anti-hCD8β single domain antibodies should not induce unwanted cytotoxicity, we next assessed the effect of the single domain antibodies on T-cell activation. To assess this, primary human peripheral blood mononuclear cells were incubated overnight with the anti-hCD8β or irrelevant single domain antibodies or CD3/CD28 dynabeads. The next day, T-cell activation was assessed via human CD69, an early immune activation marker, expression (FIG. 8A-B) and secreted IFN-γ levels (FIG. 8C). Stimulation with CD3/CD28 dynabeads resulted in significant increase of CD69 on CD8+ T-cells and secreted IFN-γ levels. In contrast, no changes in CD69 expression or secreted IFN-γ levels were observed upon incubation with anti-hCD8β single domain antibodies compared to the Irr single domain antibody. Finally, the immunogenicity of the Nbs was also determined via a dendritic cell-T-cell co culture assay using PBMCs of 30 independent healthy donors (FIG. 8D-E). As negative and positive controls, we took along clinically benchmarked Bevacizumab and immunogenic KLH protein. In contrast to KLH, none of the Nbs showed any sign of immunogenicity as no significant increase of IFN-γ or IL-5 levels were observed. Finally, the thermostability of the single domain antibodies was determined as the single domain antibodies will be incubated at higher temperatures during radiolabeling (Table 1). All three anti-hCD8β single domain antibodies displayed melting temperatures above 50° C. with single domain antibodies 22719 and 22772 showing higher melting temperatures (Tm=68.1 and 70.1° C. respectively) compared to Nb 22728 (Tm=57.1° C.).

TABLE 1
Overview of the in vitro characteristics of
the anti-human CD8b single domain antibodies.

| | k_{D} hCD8αβ | k_{D} SUP-T1 cells | k_{D} hCD8αβ | k_{D} cynomolgus | Melting |
|---|---|---|---|---|---|
| | protein (nM) | (nM) | protein | CD8β protein | temperature |
| Nb | SPR | Flow cytometry | ELISA (nM) | ELISA (nM) | (° C.) |
| 22728 | 2.6 ± 0.3 | 2.0 ± 0.4 | 2.9 | 1.7 | 57.1 ± 0.0 |
| 22719 | 1.0 ± 0.1 | 16.0 ± 9.0 | 3.6 | 4.4 | 68.1 ± 0.3 |
| 22772 | 5.0 ± 1.1 | 16.8 ± 15.5 | 3.5 | 1.6 | 70.3 ± 0.2 |

SPR: Surface plasmon resonance.

The amino acid sequence of the respective hCD8b-targeting sdAbs were determined, and their CDRs and FRs delineated.

### Amino Acid Sequence of Single Domain Antibody 22719

(SEQ ID NO: 1)
DVQLVESGGGLVQAGGSLRLSCRASGHTFSDTAVGWFRRVPGKEREFVA
TIAGSGSIRYSESVKGRFTISGDNAKNMVYLQMNRLKPEDTGVYYCAAA
VGISYDYWGQGTQVTVSS

### Amino Acid Sequence of Single Domain Antibody 22728

(SEQ ID NO: 2)
DVQLVESGGGLVQAGDSLRLSCVASGRTVSGGVMGWFRQAPGKGREFVA
SIKWESGRTYYVDSLKDGFTISRDSPKNTVYLQMTNLKPEDTAVYYCAA
GIRVWQLSTLESTYDDWGQGTQVTVSS

### Amino Acid Sequence of Single Domain Antibody 22772

(SEQ ID NO: 3)
DVQLVESGGGLVQPGGSLRLSCRASGHTFSDLSVGWFRRAPGKAREFVA
TTAWNGSIRYAESVKGRFTISRDNAANMMYLQMNSLKPEDTAIYYCAAA
VGISYDYWGQGTQVTVSS

### An Alignment of SEQ ID NOs:1-3 is Depicted Hereafter:

| 22728 | DVQLVESGGGLVQAGDSLRLSCVASGRTVSGGVMGWFRQAPGKGREFVASIKWESGRTYY | 60 |
|---|---|---|
| 22719 | DVQLVESGGGLVQAGGSLRLSCRASGHTFSDTAVGWFRRVPGKEREFVATIAGSG-SIRY | 59 |
| 22772 | DVQLVESGGGLVQPGGSLRLSCRASGHTFSDLSVGWFRRAPGKAREFVATTAWNG-SIRY | 59 |
| | ************* *.****** ***:*.*. :****:.*** *****: .. * | |
| 22728 | VDSLKDGFTISRDSPKNTVYLQMTNLKPEDTAVYYCAAGIRVWQLSTLESTYDDWGQGTQ | 120 |
| 22719 | SESVKGRFTISGDNAKNMVYLQMNRLKPEDTGVYYCAAAVG--------ISYDYWGQGTQ | 111 |
| 22772 | AESVKGRETISRDNAANMMYLQMNSLKPEDTAIYYCAAAVG--------ISYDYWGQGTQ | 111 |
| | :*:*. **** *. * :****. ******.:*****.: :** ****** | |
| 22728 | VTVSS | 125 (SEQ ID NO: 2) |
| 22719 | VTVSS | 116 (SEQ ID NO: 1) |
| 22772 | VTVSS | 116 (SEQ ID NO: 3) |
| | ***** | |

### Amino Acid Sequence of CDR and FR Regions of Single Domain Antibody 22719

CDR1:
(SEQ ID NO: 4)
GHTFSDTA
CDR2:
(SEQ ID NO: 7)
TIAGSGSIRYSE
CDR3:
(SEQ ID NO: 10)
AAAVGISYDY
FR1:
(SEQ ID NO: 15)
DVQLVESGGGLVQAGGSLRLSCRAS
FR2:
(SEQ ID NO: 20)
VGWFRRVPGKEREFVA
FR3:
(SEQ ID NO: 25)
SVKGRFTISGDNAKNMVYLQMNRLKPEDTGVYYC
FR4:
(SEQ ID NO: 30)
WGQGTQVTVSS

### Amino Acid Sequence of CDR and FR Regions of Single Domain Antibody 22772

CDR1:
(SEQ ID NO: 5)
GHTFSDLS
CDR2:
(SEQ ID NO: 8)
TTAWNGSIRYAE
CDR3:
(SEQ ID NO: 10)
AAAVGISYDY
FR1:
(SEQ ID NO: 16)
DVQLVESGGGLVQPGGSLRLSCRAS
FR2:
(SEQ ID NO: 21)
VGWFRRAPGKAREFVA
FR3:
(SEQ ID NO: 26)
SVKGRFTISRDNAANMMYLQMNSLKPEDTAIYYC
FR4:
(SEQ ID NO: 30)
WGQGTQVTVSS

### Amino Acid Sequence of CDR and FR Regions of Single Domain Antibody 22728

CDR1:
(SEQ ID NO: 6)
GRTVSGGV
CDR2:
(SEQ ID NO: 9)
SIKWESGRTYYVD
CDR3:
(SEQ ID NO: 11)
AAGIRVWQLSTLESTYDD
FR1:
(SEQ ID NO: 17)
DVQLVESGGGLVQAGDSLRLSCVAS
FR2:
(SEQ ID NO: 22)
MGWFRQAPGKGREFVA
FR3:
(SEQ ID NO: 27)
SLKDGFTISRDSPKNTVYLQMTNLKPEDTAVYYC
FR4:
(SEQ ID NO: 30)
WGQGTQVTVSS

As the CDR1 amino acid sequences of single domain antibodies 22719 and 22772 are highly identical, a common CDR1 sequence can be defined as GHTFSDXX (SEQ ID NO:12) wherein the amino acid Xaa (X) at position 7 is Thr or Leu, and the amino acid Xaa (X) at position 8 is Ala or Ser.

As the CDR1 amino acid sequences of the three single domain antibodies are highly identical, a common CDR1 sequence can be defined as GXTXSXXX (SEQ ID NO:13) wherein the amino acid Xaa (X) at position 2 is His or Arg, the amino acid Xaa (X) at position 4 is Phe or Val, the amino acid Xaa (X) at position 6 is Asp or Gly, the amino acid Xaa (X) at position 7 is Thr, Leu or Gly, and the amino acid Xaa (X) at position 8 is Ala, Ser or Val.

As the CDR2 amino acid sequences of single domain antibodies 22719 and 22772 are highly identical, a common CDR2 sequence can be defined as TXAXXGSIRYXE (SEQ ID NO:14) wherein the amino acid Xaa (X) at position 2 is lie or Thr, the amino acid Xaa (X) at position 4 is Gly or Trp, the amino acid Xaa (X) at position 5 is Ser or Asn, and the amino acid Xaa (X) at position 11 is Ser or Ala

Based on an alignment of the amino acid sequence of the FR1 regions of the single domain antibodies 22719 and 22772:

| | | | |
|---|---|---|---|
| 22719 | DVQLV ESGGG LVQAG GSLRL SCRAS | 25 | (SEQ ID NO: 15) |
| 22772 | DVQLV ESGGG LVQPG GSLRL SCRAS | 25 | (SEQ ID NO: 16) |

***** ***** *** * ***** *****,

a consensus FR1 amino acid sequence DVQLV ESGGG LVQXG GSLRL SCRAS (SEQ ID NO:18) can be construed wherein the amino acid Xaa (X) at position 14 is Ala or Pro.

Based on an alignment of the amino acid sequence of the FR1 regions of the single domain antibodies 22719, 22728 and 22772:

| | | | | |
|---|---|---|---|---|
| 22728 | FR1 | DVQLV ESGGG LVQAG DSLRL SCVAS | 25 | (SEQ ID NO: 17) |
| 22719 | FR1 | DVQLV ESGGG LVQAG GSLRL SCRAS | 25 | (SEQ ID NO: 15) |
| 22772 | FR1 | DVQLV ESGGG LVQPG GSLRL SCRAS | 25 | (SEQ ID NO: 16) |

***** ***** *** * .**** ** **,

a consensus FR1 amino acid sequence DVQLVESGGGLVQXGXSLRLSCXAS (SEQ ID NO:19) can be construed wherein the amino acid Xaa (X) at position 14 is Ala or Pro, the amino acid Xaa (X) at position 16 is Asp or Gly, and the amino acid Xaa (X) at position 23 is Val or Arg.

Based on an alignment of the amino acid sequence of the FR2 regions of the single domain antibodies 22719 and 22772:

| | | | |
|---|---|---|---|
| 22719 | VGWER RVPGK EREFV A | 16 | (SEQ ID NO: 20) |
| 22772 | VGWER RAPGK AREFV A | 16 | (SEQ ID NO: 21) |

***** *.*** **** *

a consensus FR2 amino acid sequence VGWFR RXPGK XREFV A (SEQ ID NO:23) can be construed wherein the amino acid Xaa (X) at position 7 is Val or Ala, and the amino acid Xaa (X) at position 11 is Glu or Ala.

Based on an alignment of the amino acid sequence of the FR2 regions of the single domain antibodies 22719, 22728 and 22772:

| | | | | |
|---|---|---|---|---|
| 22728 | FR2 | MGWER QAPGK GREFV A | 16 | (SEQ ID NO: 22) |
| 22719 | FR2 | VGWER RVPGK EREFV A | 16 | (SEQ ID NO: 20) |
| 22772 | FR2 | VGWER RAPGK AREFV A | 16 | (SEQ ID NO: 21) |

:**** :.*** **** *,

a consensus FR2 amino acid sequence XGWFRXXPGKXREFVA (SEQ ID NO:24) can be construed wherein the amino acid Xaa (X) at position 1 is Met or Val, the amino acid Xaa (X) at position 6 is Gln or Arg, the amino acid Xaa (X) at position 7 is Ala or Val, and the amino acid Xaa (X) at position 11 is Gly, Glu or Ala.

Based on an alignment of the amino acid sequence of the FR3 regions of the single domain antibodies 22719 and 22772:

| | | | | |
|---|---|---|---|---|
| 22719 | FR3 | SVKGR FTISG DNAKN MVYLQ MNRLK PEDTG VYYC | 34 | (SEQ ID NO: 25) |
| 22772 | FR3 | SVKGR FTISR DNAAN MMYLQ MNSLK PEDTA IYYC | 34 | (SEQ ID NO: 26) |

***** **** *** * *:*** ** ** ****. :***,

a consensus FR3 amino acid sequence SVKGR FTISX DNAXN MXYLQ MNXLK PEDTX XYYC (SEQ ID NO:28) can be construed wherein the amino acid Xaa (X) at position 10 is Arg or Gly, the amino acid Xaa (X) at position 14 is Lys or Ala, the amino acid Xaa (X) at position 17 is Val or Met, the amino acid Xaa (X) at position 23 is Arg or Ser, the amino acid Xaa (X) at position 30 is Ala or Gly, and the amino acid Xaa (X) at position 31 is Val or lie.

Based on an alignment of the amino acid sequence of the FR3 regions of the single domain antibodies 22719, 22728 and 22772:

| | | | | |
|---|---|---|---|---|
| 22728 | FR3 | SLKDG FTISR DSPKN TVYLQ MINLK PEDTA VYYC | 34 | (SEQ ID NO: 27) |
| 22719 | FR3 | SVKGR FTISG DNAKN MVYLQ MNRLK PEDTG VYYC | 34 | (SEQ ID NO: 25) |
| 22772 | FR3 | SVKGR FTISR DNAAN MMYLQ MNSLK PEDTA IYYC | 34 | (SEQ ID NO: 26) |

*:*. **** *. * :*** **** ****. :***,

a consensus FR3 amino acid sequence SXKXX FTISX DXXXN XXYLQ MXXLK PEDTX XYYC (SEQ ID NO:29) can be construed wherein the amino acid Xaa at position 2 is Leu or Val, the amino acid Xaa at position 4 is Asp or Gly, the amino acid Xaa at position 5 is Gly or Arg, the amino acid Xaa at position 10 is Arg or Gly, the amino acid Xaa at position 12 is Ser or Asn, the amino acid Xaa at position 13 is Pro or Ala, the amino acid Xaa at position 14 is Lys or Ala, the amino acid Xaa at position 16 is Thr or Met, the amino acid Xaa at position 17 is Val or Met, the amino acid Xaa at position 22 is Thr or Asn, the amino acid Xaa at position 23 is Asn, Arg or Ser, the amino acid Xaa at position 30 is Ala or Gly, and the amino acid Xaa at position 31 is Val or lie.

An sdAbs 22719 variant was synthesized wherein the lysine at position 14 in the FR3 region (position 75 in SEQ ID NO:1) was changed into alanine. This amino acid substitution did not alter binding affinity but is useful for NOTA-conjugation (NOTA is a chelator of metallic radionuclides) of the sdAb. Such variant can also be obtained for sdAb 22728 (position 14 in the FR3 region; position 76 in SEQ ID NO:2).

A tabular overview of the SEQ ID NOs associated with the CDR and FR regions of the CD8b-binding sdAbs is given hereafter. A SEQ ID NO. indicated by xx (yy-zz) means that SEQ ID NO:xx is derived from SEQ ID NOs: yy and zz as explained hereinabove.

| sdAb | | | | | | | |
|---|---|---|---|---|---|---|---|
| [SEQ ID NO] | CDR1 | CDR2 | CDR3 | FR1 | FR2 | FR3 | FR4 |
| 22719 [1] | 4 | 7 | 10 | 15 | 20 | 25 | 30 |
| 22772 [3] | 5 | 8 | 10 | 16 | 21 | 26 | 30 |
| 22728 [2] | 6 | 9 | 11 | 17 | 22 | 27 | 30 |
| | 12 (4-5) | 14 (7-8) | | 18 (15-16) | 23 (20-21) | 28 (25-26) | |
| | 13 (4-6) | | | 19 (15-17) | 24 (20-22) | 29 (25-27) | |

As the extracellular parts of the hCD8a and hCD8b proteins are extremely unrelated, it is highly unlikely for the hCD8b sdAbs identified herein to bind to hCD8a. These protein amino acid sequences are included hereinafter, and an aligned of both is included in FIG. 6.

CD8a extracellular part (amino acids 22-82 of hCD8a sequence defined by Genbank
Accesion No. NP_001139345.1):
(SEQ ID NO: 31)
SQFRVSPLDRTWNLGETVELKCQVLLSNPTSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAPTIASQPLSLRPEACRPAAGGAVHTRGLDFAC
D.
CD8b extracellular part (amino acids 22-170 of hCD8b sequence defined by Genbank
Accession No. KAI2524241.1):
(SEQ ID NO: 32)
LQQTPAYIKVQTNKMVMLSCEAKISLSNMRIYWLRQRQAPSSDSHHEFLALWDSAKGTIHGEEVEQEKIAVFRDASRF
ILNLTSVKPEDSGIYFCMIVGSPELTFGKGTQLSVVDFLPTTAQPTKKSTLKKRVCRLPRPETQKGPLCSP.

### Example 2. Anti-Human CD8b Single Domain Antibodies Target Human CD8+ T Cells in Naïve and Tumor-Bearing Human CD8 Transgenic Mice

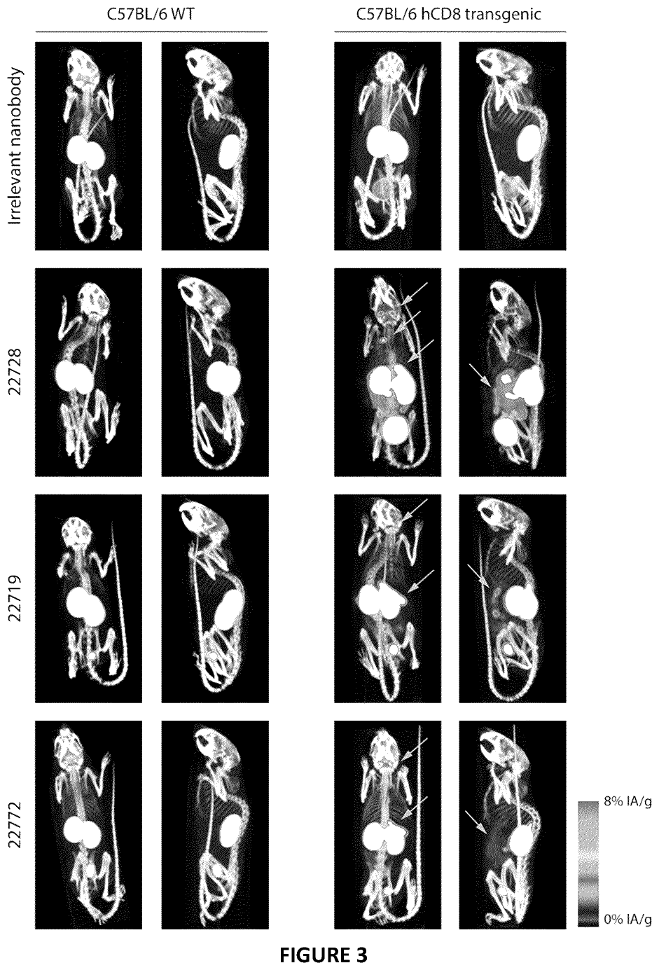

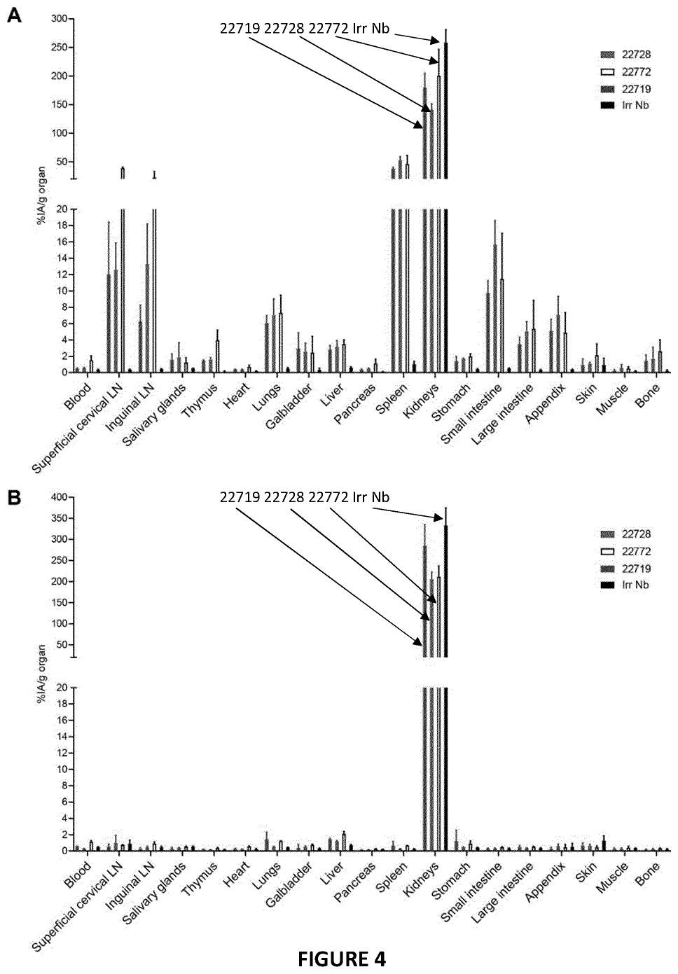

Next, we determined the potential of the anti-hCD8β single domain antibodies to target and visualize hCD8+ T cells in vivo. To this end, the anti-hCD8β single domain antibodies and the irrelevant single domain antibody were site-specifically radiolabeled with ^{99m}Tc via their C-terminal His-tag. All single domain antibodies were successfully radiolabeled with chemical purities above 99% (data not shown). First, the in vivo specificity of the ^{99m}Tc-radiolabeled single domain antibodies was assessed by SPECT/CT imaging 1 hour post intravenous injection in C57BL/6 wild type (WT) and hCD8 transgenic mice (FIG. 3). All ^{99m}Tc-radiolabeled single domain antibodies showed high uptake in the kidneys and bladder, due to rapid blood clearance, in both WT and hCD8 transgenic mice. However, the ^{99m}Tc-labeled irrelevant single domain antibody did not show any accumulation in other organs in both mice. In contrast, all three ^{99m}Tc-labeled anti-hCD8 single domain antibodies showed uptake in T-cell-rich organs such as lymph nodes, spleen, thymus and intestines of hCD8 transgenic, but not WT, mice. Interestingly, ^{99m}Tc-labeled 22728 allowed the best visualization of these organs using SPECT/CT. Specific uptake of the ^{99m}Tc-labeled anti-hCD8 single domain antibodies in hCD8 transgenic mice was confirmed via ex vivo γ-counting of the isolated organs of WT and hCD8 transgenic mice after SPECT/CT imaging (FIG. 4).

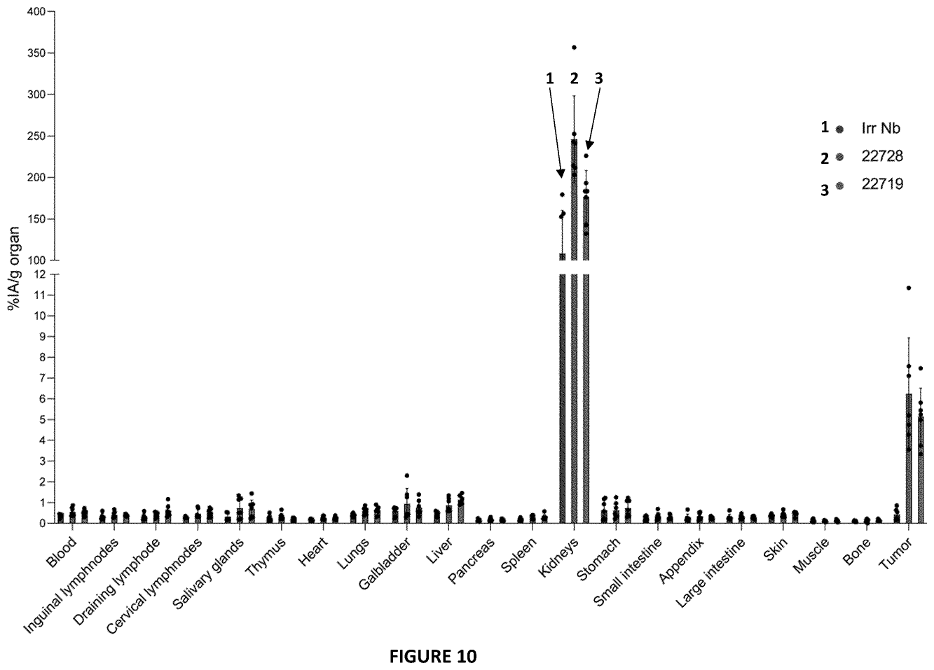

In addition, the targeting potential of the ^{99m}Tc-radiolabeled single domain antibodies was assessed in CD8+ SUP-T1 tumor-bearing immunodeficient nude mice by SPECT/CT imaging 1 hour post intravenous (i.v.) injection (FIG. 9A). All ^{99m}Tc-radiolabeled single domain antibodies showed high uptake in the kidneys and bladder, due to rapid blood clearance. However, both ^{99m}Tc-radiolabeled anti-hCD8β single domain antibodies showed uptake in the SUP-T1 tumors, which was not observed for the ^{99m}Tc-radiolabeled irrelevant single domain antibody. Specific uptake of the ^{99m}Tc-labeled anti-hCD8β single domain antibodies was further confirmed via ex vivo γ-counting of the isolated organs of SUP-T1 tumor-bearing mice after SPECT/CT imaging (FIG. 9B and FIG. 10). These data highlight the ability of ^{99m}Tc-labeled anti-hCD8β single domain antibodies to visualize endogenously expressed human CD8.

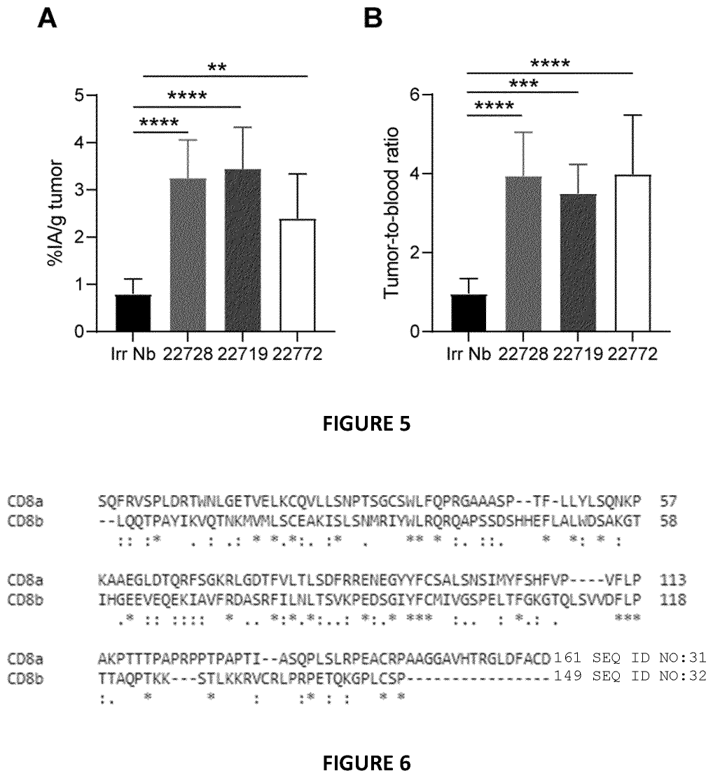

We next determined the ability of the ^{99m}Tc-labeled anti-hCD8 single domain antibodies to target intratumoral T cells (FIG. 4). To this end, hCD8 transgenic mice were subcutaneously inoculated with MC38 tumor cells and SPECT/CT imaging was performed when the tumor size reached approximately 500 mm³. As seen with the naïve hCD8 transgenic animals, no specific uptake of ^{99m}Tc-labeled irrelevant single domain antibody was observed (data not shown). Furthermore, all three ^{99m}Tc-labeled anti-hCD8 single domain antibodies showed again uptake in T-cell-rich organs (data not shown). When looking at the tumor, the ^{99m}Tc-labeled anti-hCD8 single domain antibodies showed a significantly higher uptake in the tumor compared to the ^{99m}Tc-labeled irrelevant single domain antibody (FIG. 5A). Moreover, all three ^{99m}Tc-labeled anti-hCD8 single domain antibodies showed significantly higher tumor-to-blood ratios compared to the ^{99m}Tc-labeled irrelevant single domain antibody (FIG. 5B).

### Example 3. Anti-Human CD8b Single Domain Antibodies Visualize Human CD8+ T Cell Dynamics In Vivo During Immunotherapy Treatment

Finally, we assess the possibility to follow up immunotherapy responses via visualization of CD8+ T cell dynamics. To this end, MC38 tumor-bearing hCD8 transgenic mice are treated with anti-PD-1 mAb therapy, known to induce an influx of CD8+ T cells in the tumor microenvironment, or isotype control therapy. Anti-PD-1 mAb therapy results in a reduction of tumor size, which is not the case for the control group. Imaging of the distribution of hCD8+ T cells, using the radiolabeled anti-hCD8 single domain antibodies, shows an increased uptake in the anti-PD-1 treated tumors compared to the isotype-treated tumors. These results are confirmed via ex vivo γ-counting of the tumors afterwards. Moreover, flow cytometry analysis of the tumor and tumor-draining lymph node also shows a significant increase of hCD8+ T cells in these organs of the anti-PD-1 treated animals compared to the isotype-treated group. Overall, these results show the ability of the radiolabeled anti-hCD8 single domain antibodies to visualize anti-cancer immunotherapy responses in a non-invasive manner.

### Example 4. Anti-Human CD8b PET Tracers Visualize Human CD8+ T-Cell Dynamics In Vivo

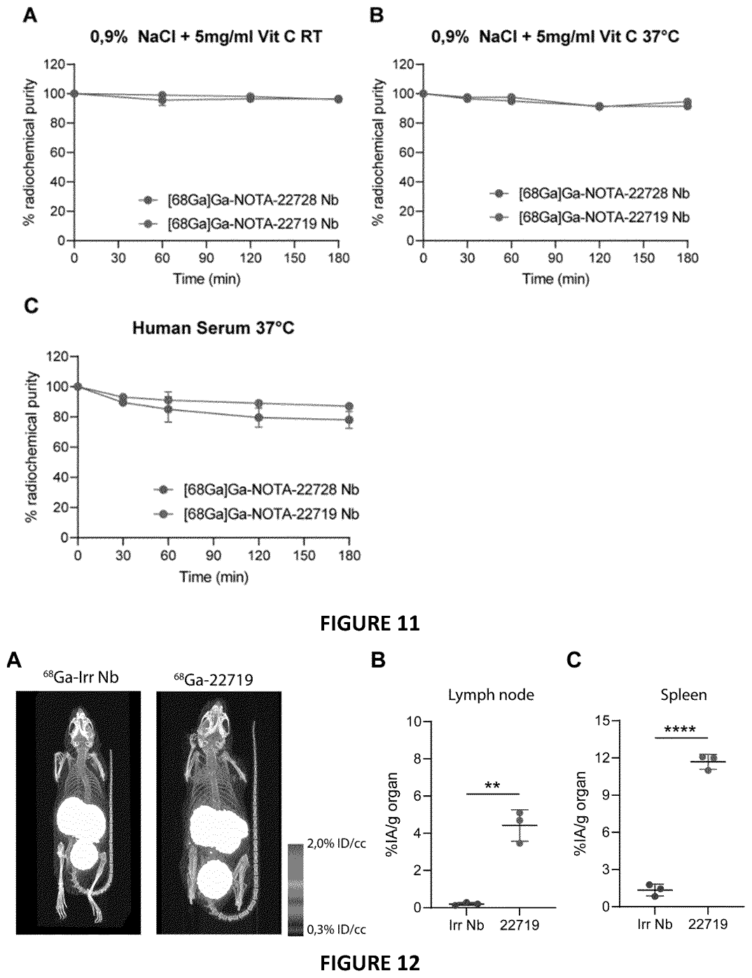

As PET imaging enables a higher spatial resolution and is more commonly used in the clinic, the anti-hCD8β single domain antibodies were converted to PET tracers. To this end, the single domain antibodies were first randomly conjugated to NOTA via their lysines and subsequently radiolabeled with Galium-68 (⁶⁸Ga). A radiochemical purity above 98% was observed even before purification for both radiolabeled anti-hCD8β single domain antibodies (data not shown). In addition, the radiolabeled anti-hCD8β single domain antibodies remained stable after incubation in injection buffer and human serum at room temperature and 37° C. (FIG. 11).

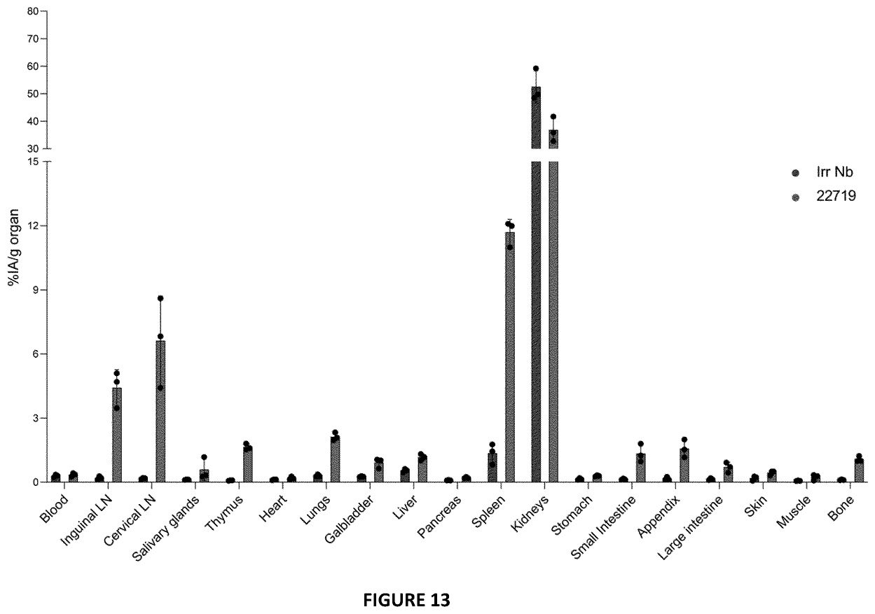

Firstly, the ability of ⁶⁸Ga-labeled anti-hCD8β single domain antibody 22719 to target hCD8+ T-cells in vivo was determined in naïve hCD8 transgenic mice. Similar to the ^{99m}Tc-labeled variant, specific uptake of ⁶⁸Ga-labeled anti-hCD8β single domain antibody 22719 in T-cell rich organs was observed (FIG. 12 and FIG. 13) via PET/CT imaging and ex vivo γ-counting.

### Example 5. Imaging with Radiolabeled Anti-Human CD8b Single Domain Antibodies Visualizes Human CD8+ T Cell Dynamics In Vivo During Immunotherapy Treatment

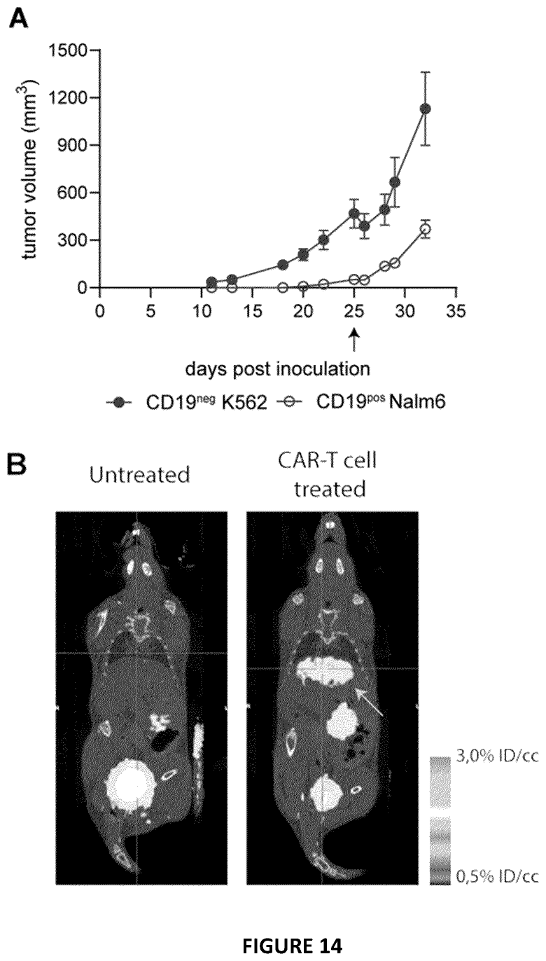

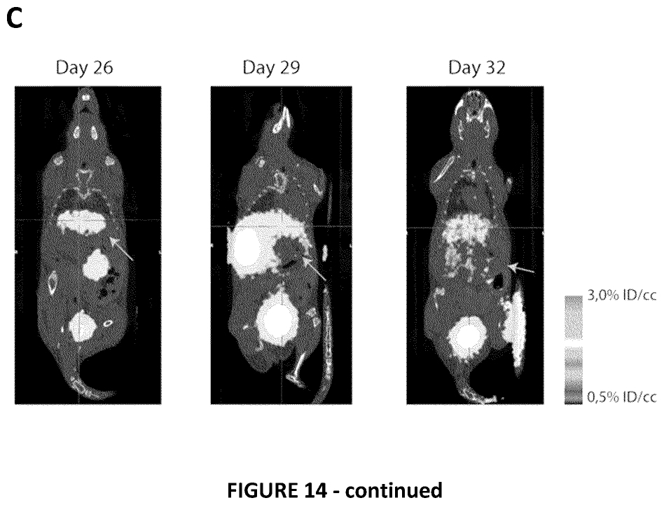

Finally, the possibility to follow up immunotherapy responses via visualization of CD8+ T cell dynamics was assessed. To this end, NSG (immunodeficient) mice were inoculated with CD19^{neg} K562 and CD19^{pos} Nalm6 tumors on each flank (tumor growth curves shown in FIG. 14 A). At day 25 post inoculation, mice were treated with PBS or human CD19-targeting CAR-T cells. Surprisingly, CAR-T cell treatment did not result in a reduction of Nalm6 tumor growth. To assess this lack of treatment effect, CAR-T cell dynamics were visualized via PET/CT imaging using the ⁶⁸Ga-labeled single domain antibody 22719 (FIG. 14 B-C). One day post CAR-T cell treatment, PET/CT imaging with the ⁶⁸Ga-labeled single domain antibody 22719 indicated the presence of the CAR-T cells in the liver as PET signal was only seen in the CAR-T cell treated mice while no signal was observed in the untreated mice (FIG. 14 B). Imaging of the CAR-T cell dynamic over a period of 1 week (day 26, 29 and 32) showed the lack of infiltration of CAR-T cells in the CD19+ Nalm6 tumors (FIG. 14 C). Only at day 7 post treatment (day 32), CAR-T cells seemed to extravasate from the liver as a lower signal is seen on the PET images. However, no uptake in Nalm6 tumors was observed. As such, PET imaging using the ⁶⁸Ga-labeled single domain antibody 22719 was able to explain the lack of therapeutic efficacy of the CAR-T cell treatment.

### Example 6. Material and Methods

### 6.1. Cell Culture

SUP-T1 cells were purchased from ATCC (Wesel, Germany). The MC38 cells line was kindly provided by Massimiliano Mazzone (VIB-KU Leuven, Belgium). Primary PBMCs of healthy volunteers were kindly provided by Karine Breckpot (Vrije Universiteit Brussels, Belgium). All cells were grown at 5% C02 and 37° C. SUP-T1 cells were grown Roswell Park Memorial Institute (RPMI) 1640 Medium (Gibco, Thermo Fisher Scientific, Waltham, Massachusetts, USA) supplemented with 1% Penicillin/Streptomycin (Gibco, Thermo Fisher Scientific) and 10% Fetal Bovine Serum (FBS, Serana, Pessin, Germany). MC38 cells were grown in Dulbecco's Modified Eagle's Medium (DMEM, Gibco, Thermo Fisher Scientific) supplemented with 1% Penicillin/Streptomycin and 10% FBS. Primary PBMCs were grown in Iscove's Modified Dulbecco's Medium (IMDM, Gibco, Thermo Fisher Scientific) supplemented with 1% Penicillin/Streptomycin, 10% Human AB serum (ZenBio, Durham, NC, USA).

### 6.2. Animal Models

Male and female wild type C57BL6/J mice and human CD8 transgenic mice (B6;SJL-Tg(CD8aCD8b)57Scr/J) and NSG mice were purchased from Charles River (Ecully, France) and Jackson laboratory (Bar Harbor, ME, USA), respectively. In the case of imaging of tumor-bearing mice, mice were subcutaneously injected with 500,000 MC38, 5 million SUP-T1, 1 million K562 or 1 million Nalm6 cells in the right or left flank. In the case of SUP-T1 cells, cells were resuspended in 50% Matrigel (Corning, Somerville, MA, USA) prior to inoculation. Mice were examined daily and tumor growth was measured using a caliper. Tumor volume was calculated using the formula (length×width²)/2. In case of administration of immunotherapy, mice are randomized in 2 groups when tumor volume reached a size of 100 mm³. Each group receives anti-PD-1 antibody (clone RMP1-14, Bio X Cell, Lebanon, NH, USA) or isotype control (clone 2A3, Bio X Cell) (250 ug per injection in 100 uL) via intraperitoneal injection every 3 days for a total amount of 4 times. In case of administration of CAR-T cells, mice were randomized in 2 groups upon the day of treatment. Each group received either PBS or 1 million of CD19-targeting CAR-T via intravenous injection. All experiments using mice were approved by the Ethical Committee for laboratory animals of the Vrije Universiteit Brussel and executed in accordance to the European guidelines for animal experimentation (ethical dossier number 21-272-1 and 23-214-20).

### 6.3. Single Domain Antibody Generation, Selection and Production

Two llamas were subcutaneously injected 6 times with 100 μg recombinant human (h)CD8β-Avi-His₆ (U-Protein Express BV, Utrecht, The Netherlands) and 100 μg recombinant human CD8β-hIgG1 Fc (Sino Biological, Eschborn, Germany) mixed with Gerbu adjuvant P (Gerbu Biotechnik, Heidelberg, Germany) on a weekly basis. After immunizations, peripheral blood of both llamas was collected and peripheral blood mononuclear cells were isolated using lymphoprep tubes (Greiner Bio-one, Kremsmunster, Austria). RNA was isolated from peripheral blood lymphocytes using a RNA extraction kit (Qiagen, Hilden, Germany) and reverse transcribed into cDNA. Next, genes coding for the variable domain of the heavy-chain only antibodies were amplified and ligated into the pMECS phage vector (Muyldermans 2021, FEBS J 288:2084-2102) resulting in 2 separate phage display libraries. Subsequent biopanning was performed by infection of the libraries with M13K07 helper phages, resulting in phage production. For each library, 3 rounds of panning in solution was performed using in-house site-specifically biotinylated hCD8β-Avi-His₆. For round 1 and 2, 100 nM of antigen was used while 10 nM of antigen was used during the final round of panning. In total, 190 unique clones (95 from round 2 and 95 from round 3) were randomly selected and screened for their ability to specifically bind to hCD8β via ELISA. Specific binding was determined via ELISA using site-specifically biotinylated hCD8β-Avi-His₆, immobilized on a streptavidin-coated 96-wells plate (Thermo Fisher Scientific). Positive hits were sent for sequencing (Eurofins genomics, Ebersberg, Germany) and grouped into different B cell lineages based on the CDR3 sequence. Single domain antibodies were produced and purified as previously described (Pardon et al. 2014, Nat Protoc 9:674-693).

### 6.4. Surface Plasmon Resonance

The affinity of purified anti-hCD8β single domain antibodies to recombinant hCD8αβ protein (R&D systems, Minneapolis, MN, USA) was determined using the BIACORE-T200 device (Cytiva, Freiburg, Germany). Surface plasmon resonance measurements were performed at 25° C. with HEPES buffered saline (HBS, 20 mM of HEPES pH 7.4, 150 mM of NaCl, 3.4 mM of EDTA 0.05% Tween-20) running buffer. The single domain antibodies were injected consecutively in 2-fold serial dilutions, from 250 to 1 nM. The association step was 100 s, the dissociation step was 200 s, and the two-step regeneration of 35 s at 30 μL·min-1, using 100 mM glycine at pH 3.0, was performed. Local curve fitting analysis was performed using the BIACORE evaluation software (Cytiva) by fitting the obtained sensorgrams to theoretical curves, assuming 1-1 binding geometries. For the determination of the equilibrium dissociation constant, the ratio of the association and dissociation rate constants was determined.

### 6.5. Affinity Determination Via Flow Cytometry

Serial dilutions of anti-hCD8β single domain antibodies were incubated with 500.000 SUP-T1 cells in FACS buffer (HBSS (Gibco, Thermo Fisher Scientific) supplemented with 1% FBS and 2 mM EDTA (Duchefa Biochemie, Haarlem, The Netherlands)) for 1 h at 4° C. Cells were washed once with FACS buffer. Next, single domain antibody binding was detected by incubation of the cells with an Alexa Fluor®-488 tagged anti-HA antibody (1:1000 in FACS buffer, clone 16B12, Biolegend, San Diego, CA, USA) for 30 min at 4° C. Again, cells were washed once with FACS buffer. Single domain antibody binding was determined using the FACS CANTO II analyser (BD Biosciences, Franklin Lakes, NJ, USA). The mean fluorescence intensity of single domain antibody binding was determined using FlowJo version 10.

### 6.6. Affinity Determination Via ELISA

Wells of a 96 well MicroWell MaxiSorp flat bottom plate (Thermo Fisher Scientific) were coated with 0.2 μg of recombinant hCD8αβ protein, 0.2 μg of cynomolgus CD8β-Fc protein (Sino Biologicals) or PBS overnight at 4° C. The next day, wells were washed 3 times with PBS-T (PBS+0.05% Tween20 (Merck-Millipore, Burlington, MA, USA). Next, wells were blocked with blocking buffer (2% Skimmed milk (Regilait) in PBS) for 1 h at room temperature (RT). Different concentrations of single domain antibodies, diluted in blocking buffer, were added to the wells and incubated for 1 h at RT. Single domain antibody binding was detected using a mouse-anti-HA antibody (1:2000, clone 16B12, Biolegend) and alkaline-phosphatase conjugated goat-anti-mouse antibody (1:200, clone A90-116AP, Bethyl Laboratories, Montgomery, TX, USA). Wells were washed 5 times with PBS-T between all incubation steps. Binding was determined using p-nitrophenyl phosphate (2 mg/ml resuspended in AP blot buffer (12.12 g/L Trizma base, 10.17 g MgCl2·6H20/L, 5.84 g/L NaCl); Thermo Fisher Scientific). Absorbance at 405 nm was measured via a VersaMax ELISA Microplate Reader, using the SoftMax® Pro software (Molecular Devices, San Jose, CA, USA).

### 6.7. Thermal Shift Assay

Single domain antibodies (concentration ranging 0.2 mg/ml to 0.5 mg/ml) were mixed with 1× SYPRO™ Orange Protein Gel Stain (Thermo Fisher Scientific) in PBS and added to white 96-well PCRs plates (Biorad, Pleasanton, CA, USA). Fluorescence signal was measured during increasing temperature steps ranging from 20 to 95° C., with stepwise increments of 0.5° C., using CFX Connect™ Real-Time PCR (Biorad). Melting temperatures of the single domain antibodies was calculated using the Boltzmann equation.

### 6.8. Single Domain Antibody Binding to Primary T Cells

One day prior to the analysis, primary lymphoid cells were thawed and taken into culture. The next day, cells were resuspended in HBSS and 500.000 cells were taken for each sample. Cells were stained with eBioscience™ Fixable Viability Dye eFluor™ 506 (1:1000 in HBSS; Thermo Fisher Scientific) for 30 min at 4° C. Cells were washed once with FACS buffer. Next, samples were incubated with human FcR blocking agent (Miltenyi Biotec, Bergisch Gladbach, Germany) diluted in FACS buffer according to manufacturers' protocol for 10 min at 4° C. Next, 100 nM of anti-hCD8α (clone R3HCD27, patent US20190071500A1), anti-hCD8β (current invention) or irrelevant single domain antibodies were added for 1 h at 4° C. Cells were washed once with FACS buffer and incubated with a mix of fluorescent antibodies (Table 2) for 30 min at 4° C. Cells were washed once again with FACS buffer before single domain antibody binding was determined using the FACS CANTO II analyser. Analysis of the single domain antibody binding was performed using FlowJo version 10.

TABLE 2
Overview antibodies used for flow cytometry

| Target | Fluorophore | Species reactivity | Provider | Clone |
|---|---|---|---|---|
| CD11b | PE/Cyanine7 | Human, Mouse | Biolegend | M1/70 |
| His-tag | APC | / | Miltenyi Biotec | GG11-8F3.5.1 |
| CD8 | APC | Human | BD Bioscience | 2ST8.5H7 |
| CD8 Isotype | APC | / | BD Bioscience | G155-178 |
| control | | | | |
| CD4 | PerCP/Cyanine5.5 | Human | Biolegend | RPA-T4 |
| CD4 | PerCP/Cyanine5.5 | Mouse | Biolegend | GK1.5 |
| CD56 | PerCP/Cyanine5.5 | Human | Biolegend | QA18A21 |
| CD3 | FITC | Human | eBioscience | SK7 |
| TCR beta | FITC | Mouse | eBioscience | H57-597 |
| CD19 | PE | Human | Biolegend | SJ25C1 |
| CD19 | PE | Mouse | eBioscience | 1D3 |
| CD45 | APC/Cyanine7 | Human | Biolegend | HI30 |
| CD45 | APC/Cyanine7 | Mouse | Biolegend | 30-F11 |
| CD8 | Brilliant violet 421 | Mouse | Biolegend | 53-6.7 |
| CD69 | APC | Human | Biolegend | FN50 |
| CD8 | Brilliant violet 421 | Human | Biolegend | RPA-T8 |

6.9. ^{99m}Tc-Radiolabeling of Single Domain Antibodies

Single domain antibodies were labeled with ^{99m}Tc as previously described (Xavier et al. 2012, Methods Mol Biol 911:485-490). Briefly, ^{99m}Tc-tricarbonyl was generated via the addition of 150 mCi ^{99m}TcO₄⁻ to the Isolink® labelling kit (Paul Scherrer Institute, Villigen, Switzerland) for 20 min at 100° C. Next, 50 μg of His-tagged single domain antibody was added and incubated for 90 min at 37 or 50° C. ^{99m}Tc-labeled single domain antibodies were purified via gel filtration from the unbound [^{99m}(H2O)₃(CO)₃]+ via a NAP-5 column (Cytiva) and filtered through a Millex 0.22 μm filter (Millipore, Haren, Belgium). The radiochemical purity of radiolabeled single domain antibodies was evaluated by instant thin layer chromatography (iTLC, Pall Corporation, Hoegaarden, Belgium)

### 6.10. Pinhole SPECT-Micro-CT Imaging and Image Analysis

Mice were injected with 5 μg of radiolabeled single domain antibody. One hour post injection, mice were anesthetized with 75 mg/kg ketamine and 1 mg/kg medetomidine (Ketamidor, Richter Pharma AG, Weis, Austria) via intraperitoneal injection and SPECT/micro-CT Imaging was performed using a Vector⁺ scanner (MiLABS, Houten, The Netherlands). Imaging set-up consisted of a 1.5 mm 75-pinhole general purpose collimator, in spiral mode with 6 bed positions. Total SPECT scanning time was 15 minutes with 150 seconds per position and CT scanning (60 kV and 615 mA) was 2 minutes. After imaging, mice were euthanized and organs were collected. Radioactivity in each organ was determined using a Wizard² γ-counter (Perkin-Elmer, Waltham, MA, USA). Uptake in each organ was corrected for radioactive decay and calculated as percentage of injected activity per gram of organ. SPECT/CT image analysis was performed using AMIDE (UCLA, CA, USA) and OsiriX (Pixmea, Geneva, Switzerland) software.

### 6.11. Processing Organs and Flow Cytometry Analysis

Single cell preparations of tumors and tumor-draining lymph nodes are prepared as described previously (Van Damme et al. 2021, J Immunother Cancer 9: e001749). Antibodies used for staining of single cell preparations can be found in Table 2. Delta median fluorescence intensity (AMFI) was determined via subtraction of the MFI of the staining and the MFI of the isotype control. Data was acquired using the FACS CANTO II analyser and analyzed using FlowJo.

### 6.12. NOTA-Conjugation of Single Domain Antibodies

The random conjugation of the single domain antibodies to p-SCN-Bn-NOTA (NOTA-NCS, Macrocyclics, Inc., Plano, TX, USA) was based on the standard protocol previously described with some adaptations (Xavier et al. 2013, J Nucl Med 54:776-784). The single domain antibodies were first buffer-exchanged to 0.25 M sodium carbonate adjusted to pH 9.1 (sodium carbonate anhydrous; sodium hydrogen carbonate; sodium chloride, VWR Chemicals, Leuven, Belgium) using a PD-10 size exclusion column (GE Healthcare, Buckinghamshire, UK). A 20-fold molar excess of NOTA-NCS was added to the single domain antibody solution and incubated for 2h30 at RT. After incubation, the NOTA-single domain antibody was purified via size exclusion chromatography (SEC) on a Hiload™ 16/600 Superdex™ 30 pg column (GE Healthcare Bio-Sciences AB, Uppsala, Sweden) with 0.1 M NaOAc as a mobile phase (0.8 mL/min) to separate the conjugated single domain antibody from excess NOTA-NCS. The concentrations of the collected NOTA-single domain antibody fractions were measured spectrophotometrically using a Nanodrop 2000 by UV absorption at 280 nm. In addition, SEC with a Superdex Peptide 10/300 GL column (GE Healthcare Bio-Sciences AB, Uppsala, Sweden) was performed for quality control of the NOTA-single domain antibody. The number of chelates per single domain antibody was determined by electrospray ionization quadrupole time-of-flight mass spectrometry (ESI-Q-TOF-MS). After determining the chelator-to-single domain antibody ratio, anion exchange chromatography (AEX) was performed using an ENrich Q 5×50 column (Bio-Rad Laboratories, Inc., California, CA, USA) with 0.02 M Tris (VWR Chemicals, Leuven, Belgium) adjusted to pH 7.5 as solvent A and 0.02 M Tris with 0.31 M NaCl as solvent B (1.5 mL/min) to separate the fractions with different chelator-to-single domain antibody ratios. The fractions with a 1:1 chelator-to-single domain antibody ratio was used for further radiolabeling.

### 6.13. ⁶⁸Ga-Radiolabeling of Single Domain Antibody

The randomly conjugated NOTA-conjugated single domain antibody (7.8 nmol for Nb 22719-NOTA and 7.1 nmol for single domain antibody 22728-NOTA) was added to 1 mL of 1 M NaOAc buffer pH 5 and 1 mL of Gallium-68 (68Ga) eluate (424-636 MBq) eluted from a 68Ge/68Ga generator in 0.1 M HCl (Galli Eo™, IRE ELiT, Fleurus, Belgium) and incubated for 10 min at RT. Purification was performed on a PD-10 desalting column pre-equilibrated with 1× PBS in case of test-labeling or 0.9% NaCl containing 5 mg/mL vitamin C pH 5.8-6.1 (injection buffer) in case of stability and in vivo studies. After purification, the radioactive single domain antibody solution was filtered through a 0.22 μm filter (Millipore, Belgium). The radiochemical purity was assessed before and after purification by radio-iTLC ([68Ga]Ga-NOTA-single domain antibody Rf=0, [68Ga]Ga-citrate Rf=1). Radiometal chelation stability of the radiolabeled single domain antibody was assessed in different conditions (injection buffer (0.9% NaCl+5 mg/ml Vitamin C) at RT, 37° C.; human serum 37° C.) at 30 min, 60 min, 120 min and 180 min after labeling. Stability of the radiolabeled compound was analyzed via radio-iTLC and radio-SEC at these timepoints

### 6.14. PET-CT Imaging and Image Analysis

Mice were injected (i.v.) with 5 μg of radiolabeled single domain antibody (±10 mBq). One hour post injection, mice were anesthetized with 75 mg/kg ketamine and 1 mg/kg medetomidine via intraperitoneal injection or isoflurane (5% induction, 2.5% maintenance, oxygen flow rate between 0.3 and 1.5 L/min; Virbac, Nice, France) via inhalation and PET/CT Imaging was performed (MoleCubes, Gent, Belgium). PET scans of 12-20 min were performed followed by a CT scan. After imaging, mice were euthanized and organs were collected. Radioactivity in each organ was determined using a Wizard² γ-counter (Perkin-Elmer). Uptake in each organ was corrected for radioactive decay and calculated as percentage of injected activity per gram of organ. PET/CT image analysis was performed using VivoQuant (Invicro, Needham, MA, USA) or OsiriX software.

1. A polypeptide binding to human CD8b, wherein the amino acid sequence of the polypeptide is comprising a CDR1 region, a CDR2 region, and a CDR3 region, wherein the CDR1, CDR2 and CDR3 regions are selected from those CDR1, CDR2 and CDR3 regions as present in an immunoglobulin variable domain (IVD) defined by SEQ ID NOs:1, 2 or 3 and as determined by the Kabat, Chothia, Martin, or IMTG method.

2. The polypeptide according to claim 1 wherein the CDR1 region is chosen from SEQ ID NO: 12 and 13, the CDR2 region is chosen from SEQ ID NOs: 14 and 9, and the CDR3 region is chosen from SEQ ID NOs: 10 and 11.

3. The polypeptide according to claim 1 further comprising at least an FR1, FR2, FR3, or FR4 region as present in an IVD.

4. The polypeptide according to claim 3 wherein the FR1 region is defined by SEQ ID NO: 19, the FR2 region is defined by SEQ ID NO:24, the FR3 region is defined by SEQ ID NO:29, and the FR4 region is defined by SEQ ID NQ:30.

5. The polypeptide according to claim 1 wherein the CDR and/or FR regions are humanized and/or the IVD is humanized.

6. The polypeptide according to claim 1 which is further comprising a functional moiety.

7. The polypeptide according to claim 6 wherein the functional moiety is a His-tag or a peptide motif recognized by a peptide ligase.

8. The polypeptide according to claim 6 wherein the functional moiety is a detectable moiety.

9. The polypeptide according to claim 8 wherein the detectable moiety is linked to a specific site comprised in the polypeptide.

10. An isolated nucleic acid encoding a polypeptide according to claim 1.

11. A vector comprising the nucleic acid according to claim 10.

12. A host cell expressing a polypeptide according to claim 1, or comprising a nucleic acid encoding said polypeptide, or comprising a vector comprising said nucleic acid.

13. A pharmaceutical composition comprising a polypeptide according to claim 1.

14. A method of diagnosis, said method comprising administration of the polypeptide according to claim 1, or a pharmaceutical composition comprising said polypeptide to a subject.

15. A method for producing a polypeptide according to claim 1 comprising:
expressing the polypeptide in a host cell comprising a nucleic acid encoding the polypeptide or a vector comprising said nucleic acid, or synthetic manufacture of the polypeptide;
purifying the expressed or manufactured polypeptide; and
optionally, coupling a detectable moiety to the purified polypeptide.

16. A method of therapy monitoring, said method comprising administration of the polypeptide according to claim 1, or a pharmaceutical composition comprising said polypeptide to a subject.

17. A method of surgical resection of a tumor, said method comprising administration of the polypeptide according to claim 1, or a pharmaceutical composition comprising said polypeptide, to a subject, optionally wherein the polypeptide comprises a detectable moiety and assists in delineating the tumor during resection.

18. An imaging agent comprising the polypeptide according to claim 1, or a pharmaceutical composition comprising said polypeptide.
